(19) United States
(12) Patent Application Publication
(10) Pub. No.: US 2021/0323929 A1
(43) Pub. Date: Oct. 21, 2021

(54) Purified Detomidine, Process of Preparing and Methods of Use

(71) Applicant: Clexio Biosciences Ltd., Jerusalem (IL)

(72) Inventors: Anton Frenkel, Jerusalem (IL); Vladimir Ioffe, Jerusalem (IL)

(21) Appl. No.: 17/148,327

(22) PCT Filed: Jul. 18, 2019
(86) PCT No.: PCT/IB2019/056160

§ 371 Date: Jan. 13, 2021

Related U.S. Application Data
(60) Provisional application No. 62/700,067, filed on Jul. 18, 2018.

(51) Int. Cl. C07D 233/58; G01N 33/15

(52) U.S. Cl. CPC C07D 233/58; C07B 2200/13; G01N 33/15

(57) ABSTRACT

The present disclosure relates to a purified detomidine HCl pharmaceutical product and to methods of preparation, validation of pharmaceutically acceptable product and use thereof.

### CROSS REFERENCE TO RELATED APPLICATIONS

This application is a continuation-in-part of International Application No. PCT/IB2019/056160, filed Jul. 18, 2019, and claims the benefit of U.S. Provisional Application No. 62/700,067, filed Jul. 18, 2018, the entireties of which are incorporated by reference herein.

### TECHNICAL FIELD

The present disclosure relates to a purified detomidine HCl pharmaceutical product and to methods of preparation, validation of pharmaceutically acceptable product and use thereof.

### BACKGROUND

Detomidine

Detomidine, 4-[(2,3-dimethylphenyl)methyl]-1H-Imidazole, is an α-2-andregenic agonist available under the brand name Equimidine® and Dormosedan® for use as a veterinary sedative. Detomidine is not currently approved for human use.

Detomidine and related compounds, including its 3,4 dimethyl isomer, iso-detomidine (4-(3,4-Dimethylbenzyl)-1H-imidazole) were first described in U.S. Pat. No. 4,443,466. Both the '466 patent and the later U.S. Pat. No. 4,584,383 describe the synthetic method of manufacturing detomidine as being based on coupling of an imidazole moiety with 1-Bromo-2,3-dimethyl benzene using a Grignard reaction. RU2448095 describes an alternative route of synthesis of the molecule based on coupling in presence of a Titanium catalyst. According to both the '383 and '095 patents, detomidine is purified by crystallization of its hydrochloride salt from water. The chemical structures of detomidine HCl and iso-detomidine are shown below:

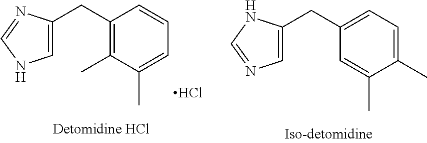

Two solid state forms of detomidine HCl are known, the anhydrous and monohydrate forms. Synthesis of the anhydrous form by crystallization of the monohydrate and further decomposition at elevated temperatures is described in U.S. Pat. No. 7,728,147. Synthesis of the anhydrous form via decomposition of the monohydrate in reduced pressure is described in Laine et al (1983). According to Veldre et al (2011), the anhydrous and monohydrate forms of detomidine HCl can easily interconvert depending on temperature and humidity.

The European Pharmacopeia 9.0 monograph (January 2014) describes detomidine HCl for veterinary use. The monograph lists the established HPLC method for identification of detomidine and its impurities as using a Symmetry C8, 5 μm, 4.6×150 mm column, with a mobile phase of Ammonium phosphate buffer pH 7.9—65% and Acetonitrile—35% at a flow rate of 1.0 mL/min and UV detection at 220 nm. That procedure is listed as recording three distinct impurities of detomidine:

### Impurity A: (RS)-(2,3-dimethylphenyl)(1H-imidazol-4-yl)methanol

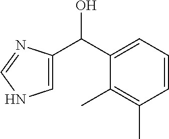

### Impurity B: (RS)-(1-benzyl-1H-imidazol-5-yl)(2,3-dimethylphenyl)methanol

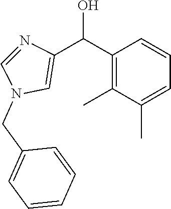 [SMILES: Cc1cccc(C(O)c2cn(Cc3ccccc3)cn2)c1C]

### Impurity C: 4-[(2,3-dimethylcyclohexyl)methyl]-1H-imidazole

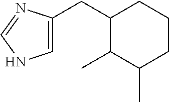

PCT/US18/012579 discloses topical formulations of detomidine and their uses in treating pain.

Purified detomidine for use in human pharmaceutical formulations is not known in the art.

### SUMMARY OF THE DISCLOSURE

It has now been identified that commercially available detomidine HCl products contain not only Impurity A (RS)-(2,3-dimethylphenyl)(1H-imidazol-4-yl)methanol), and potentially Impurities B (RS)-((1-benzyl-1H-imidazol-5-yl)(2,3-dimethylphenyl)methanol) and C (4-[(2,3-dimethylcyclohexyl)methyl]-1H-imidazole), but also significant quantities of iso-detomidine and/or iso-impurity A ((RS)-(3,4-dimethylphenyl)(1H-imidazol-4-yl)methanol) and/or 3-((1H-Imidazol-4-yl)methyl)-2-methylbenzaldehyde. Provided herein are the compound, detomidine HCl substantially free of impurities, including isomeric impurities of detomidine, compositions comprising same, a process for identifying the impurities and a novel processes for the manufacture of purified detomidine HCl.

The subject invention provides detomidine or a pharmaceutically acceptable salt thereof; the compound substantially free of iso-detomidine and/or iso-impurity A:

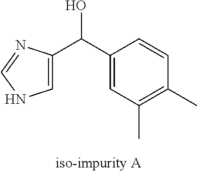

and/or 3-((1H-Imidazol-4-yl)methyl)-2-methylbenzaldehyde (“aldehyde impurity”):

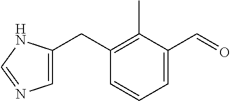

### 3-((1H-Imidazol-4-yl)methyl)-2-methylbenzaldehyde (“Aldehyde Impurity”)

In some embodiments the detomidine or a pharmaceutically acceptable salt thereof, e.g. detomidine HCl, is substantially free of iso-detomidine and iso-impurity A ((RS)-(3,4-dimethylphenyl)(1H-imidazol-4-yl)methanol).

In some embodiments the detomidine or a pharmaceutically acceptable salt thereof, e.g. detomidine HCl, is substantially free of iso-detomidine and 3-(1H-Imidazol-4-yl)methyl)-2-methylbenzaldehyde. In some embodiments the detomidine or a pharmaceutically acceptable salt thereof, e.g. detomidine HCl, is substantially free of iso-detomidine, iso-impurity A ((RS)-(3,4-dimethylphenyl)(1H-imidazol-4-yl)methanol) and 3-((1H-Imidazol-4-yl)methyl)-2-methylbenzaldehyde.

In some embodiments, the pharmaceutically acceptable salt of detomidine is detomidine HCl. In some embodiments the detomidine HCl is the monohydrate form. In some embodiments the detomidine HCl is the anhydrate form.

In some embodiments, the total amount of impurities is not more than 0.1% area, not more than 0.06% area, or not more than 0.02% area, relative to detomidine, based on HPLC, using UV detection at 220 nm. In some embodiments, the total amount of impurities is not more than 0.06% area, relative to detomidine, based on HPLC, using UV detection at 220 nm.

Further provided is a composition, optionally a pharmaceutical composition, comprising detomidine or a pharmaceutically acceptable salt thereof, substantially free of iso-detomidine and/or iso-impurity A and/or 3-((1H-Imidazol-4-yl)methyl)-2-methylbenzaldehyde. In some embodiments of the composition or pharmaceutical composition, the pharmaceutically acceptable salt of detomidine is detomidine HCl. In some embodiments of the composition or pharmaceutical composition, the detomidine HCl is the monohydrate form or the anhydrate form. In some embodiments of the composition or pharmaceutical composition, the detomidine is substantially free of iso-detomidine. In some embodiments of the composition or pharmaceutical composition, the detomidine is substantially free of iso-detomidine and iso-impurity A ((RS)-(3,4-dimethylphenyl)(1H-imidazol-4-yl)methanol). In some embodiments of the composition or pharmaceutical composition, the detomidine is substantially free of iso-detomidine and 3-((1H-Imidazol-4-yl)methyl)-2-methylbenzaldehyde. In some embodiments of the composition or pharmaceutical composition, the detomidine is substantially free of iso-detomidine, iso-impurity A ((RS)-(3,4-dimethylphenyl)(1H-imidazol-4-yl)methanol) and 3-(1H-Imidazol-4-yl)methyl)-2-methylbenzaldehyde.

In some embodiments the detomidine or a pharmaceutically acceptable salt thereof, e.g. detomidine HCl, is further substantially free of iso-impurity A (((RS)-(3,4-dimethylphenyl)(1H-imidazol-4-yl)methanol)). In some embodiments the detomidine or a pharmaceutically acceptable salt thereof, e.g. detomidine HCl, is further substantially free of impurity A ((RS)-(2,3-dimethylphenyl)(1H-imidazol-4-yl)methanol). In some embodiments detomidine or a pharmaceutically acceptable salt thereof, e.g. detomidine HCl, is further substantially free of 3-((1H-Imidazol-4-yl)methyl)-2-methylbenzaldehyde. In some embodiments, the detomidine or a pharmaceutically acceptable salt thereof, e.g. detomidine HCl, is further substantially free of impurity B ((RS)-(1-benzyl-1H-imidazol-5-yl)(2,3-dimethylphenyl)methanol) and/or impurity C (4-[(2,3-dimethylcyclohexyl)methyl]-1H-imidazole).

In some embodiments, the total amount of impurities in the detomidine or a pharmaceutically acceptable salt thereof, e.g. detomidine HCl, is not more than 0.1% area, not more than 0.06% area, or not more than 0.02% area, relative to detomidine, based on HPLC, using UV detection at 220 nm. In some embodiments, the total amount of impurities is not more than 0.06% area, relative to detomidine, based on HPLC, using UV detection at 220 nm.

The subject invention also provides a composition comprising

a) an amount of detomidine or a pharmaceutically acceptable salt thereof;

b) a vehicle; and optionally

c) an impurity which is iso-detomidine and/or iso-impurity A and/or 3-((1H-Imidazol-4-yl)methyl)-2-methylbenzaldehyde,

wherein the impurity is present in an amount no more than 0.01% area relative to the detomidine, based on HPLC, using UV detection at 220 nm.

The subject invention also provides a pharmaceutical composition comprising

a) an amount of detomidine or a pharmaceutically acceptable salt thereof;

b) at least one pharmaceutically acceptable carrier; and optionally

c) an impurity which is iso-detomidine and/or iso-impurity A and/or 3-((1H-Imidazol-4-yl)methyl)-2-methylbenzaldehyde,

wherein the impurity is present in an amount no more than 0.01% area relative to the detomidine, based on HPLC, using UV detection at 220 nm.

In some embodiments of the composition or pharmaceutical composition, the detomidine is detomidine HCl. In some embodiments the detomidine HCl is the monohydrate form of detomidine HCl. In some embodiments the detomidine HCl is the anhydrous form of detomidine HCl. In some embodiments the composition or pharmaceutical composition is substantially free of iso-detomidine. In some embodiments the composition or pharmaceutical composition is substantially free of iso-detomidine and iso-impurity A. In some embodiments, the composition or pharmaceutical composition is substantially free of iso-detomidine, iso-impurity A and 3-((1H-Imidazol-4-yl)methyl)-2-methylbenzaldehyde. In some embodiments, the composition or pharmaceutical composition is substantially free of iso-impurity A and 3-((1H-Imidazol-4-yl)methyl)-2-methylbenzaldehyde. In some embodiments, the composition or pharmaceutical composition is substantially free of iso-detomidine and 3-((1H-Imidazol-4-yl)methyl)-2-methylbenzaldehyde.

In some embodiments of the composition or the pharmaceutical composition, the detomidine or a pharmaceutically acceptable salt thereof, e.g. detomidine HCl, is further substantially free of iso-impurity A (((RS)-(3,4-dimethylphenyl)(1H-imidazol-4-yl)methanol)).

In some embodiments the composition or the pharmaceutical composition, the detomidine or a pharmaceutically acceptable salt thereof, e.g. detomidine HCl, is further substantially free of impurity A ((RS)-(2,3-dimethylphenyl)(1H-imidazol-4-yl)methanol).

In some embodiments of the composition or the pharmaceutical composition, the detomidine or a pharmaceutically acceptable salt thereof, e.g. detomidine HCl, is further substantially free of 3-((1H-Imidazol-4-yl)methyl)-2-methylbenzaldehyde.

In some embodiments of the composition or the pharmaceutical composition, the detomidine or a pharmaceutically acceptable salt thereof, e.g. detomidine HCl, is further substantially free of impurity B ((RS)-(1-benzyl-1H-imidazol-5-yl)(2,3-dimethylphenyl)methanol) and/or impurity C (4-[(2,3-dimethylcyclohexyl)methyl]-1H-imidazole).

The present invention further provides a process for the preparation of a monohydrate form of detomidine HCl substantially free of iso-detomidine comprising
a. crystallizing a monohydrate form of detomidine HCl from an aqueous solution of an anhydrous form of detomidine HCl which comprises iso-detomidine; wherein the crystallization takes place by cooling the aqueous solution to a temperature of from about 0° C. to 31° C., optionally in the presence of solid detomidine base or detomidine HCl, until a slurry is formed, and
b. collecting the monohydrate form of detomidine HCl substantially free of iso-detomidine.

Further provided is a process for the preparation of a monohydrate form of detomidine HCl substantially free of iso-detomidine comprising
a. treating an aqueous solution of an anhydrous form of detomidine HCl which comprises iso-detomidine with active carbon;
b. converting the detomidine HCl to detomidine base to form an aqueous solution of detomidine base;
c. crystallizing the monohydrate form of detomidine HCl from the aqueous solution of detomidine base by adding a sufficient amount of HCl, wherein the crystallization takes place by cooling the aqueous solution to between about 3° C. and 37° C., optionally in the presence of solid detomidine HCl, until a slurry is formed, and
d. collecting the monohydrate form of detomidine HCl substantially free of iso-detomidine.

In some embodiments of the processes, the HCl:detomidine base ratio (mole:mole) is about 1 to about 1.5, preferably about 1.5. In some embodiments, the water:detomidine (V/wt) HCl ratio is about 2-3.

In various embodiments, the crystallization of the monohydrate form of detomidine HCl from an aqueous solution occurs at a dissolution temperature of between about 35° C. and 50° C. In some embodiments, the crystallization initiation temperature is about 30° C. to 45° C.

In some embodiments of the processes, the monohydrate form of detomidine HCl has a mean crystal size of 0.3 to 0.7 mm. In some embodiments, the shape of the crystals of the monohydrate form of detomidine HCl is rod like and/or prism like. The monohydrate form of detomidine HCl has a water content of about 7.5% as determined by Karl Fisher analysis.

Further provided is the monohydrate form of detomidine HCl obtained by the processes disclosed herein, and compositions and pharmaceutical compositions comprising same; and optionally a carrier.

Further provided is use of a pharmaceutical composition comprising detomidine or a pharmaceutically acceptable salt thereof, substantially free of iso-detomidine; optionally further substantially free of the impurity A ((RS)-(2,3-dimethylphenyl)(1H-imidazol-4-yl)methanol), 3-((1H-Imidazol-4-yl)methyl)-2-methylbenzaldehyde, impurity B ((RS)-(1-benzyl-1H-imidazol-5-yl)(2,3-dimethylphenyl)methanol) and/or impurity C (4-[(2,3-dimethylcyclohexyl)methyl]-1H-imidazole).

Further provided is a method of treating a human subject in need thereof, comprising administering to the subject a pharmaceutical composition comprising detomidine or a pharmaceutically acceptable salt thereof, substantially free of iso-detomidine; optionally further substantially free of the impurity A ((RS)-(2,3-dimethylphenyl)(1H-imidazol-4-yl)methanol), 3-((1H-Imidazol-4-yl)methyl)-2-methylbenzaldehyde, impurity B ((RS)-(1-benzyl-1H-imidazol-5-yl)(2,3-dimethylphenyl)methanol) and/or impurity C (4-[(2,3-dimethylcyclohexyl)methyl]-1H-imidazole). In some embodiments the human subject is in need of an analgesic.

The subject invention yet further provides a process for producing a validated batch of a drug substance containing detomidine or a pharmaceutically acceptable salt thereof and at least one pharmaceutically acceptable carrier for distribution, the process comprises:

a) obtaining a batch of the drug substance;

b) determining by apparatus the total amount of iso-detomidine and/or iso-impurity A and/or 3-((1H-Imidazol-4-yl)methyl)-2-methylbenzaldehyde in a sample of the batch; and

c) validating the batch for distribution only if the sample of the batch is determined by weight of detomidine to contain less than 0.01% area of iso-detomidine and/or iso-impurity A and/or 3-((1H-Imidazol-4-yl)methyl)-2-methylbenzaldehyde relative to detomidine based on HPLC, using UV detection at 220 nm.

### BRIEF DESCRIPTION OF THE DRAWINGS

FIG. 1: HPLC chromatogram showing impurity in sourced samples of detomidine HCl.

FIG. 2: HPLC chromatogram showing identification of the impurity peaks

FIG. 3 LC-MS/MS identifying one of the impurity peaks as iso-impurity A.

FIG. 4 HR-ESI-MS identifying one of the impurity peaks as 3-((1H-Imidazol-4-yl)methyl)-2-methylbenzaldehyde.

FIG. 5 is a DVS isotherm plot showing hydration of detomidine free base at 24.4° C.

FIG. 6 is a DVS isotherm plot showing hydration of detomidine HCl at 24.7° C.

FIG. 7 a microphotograph of particle morphology of detomidine HCl monohydrate (sample 3).

FIG. 8 is a XRPD pattern of detomidine HCl monohydrate (sample 1).

FIG. 9 is a DSC thermogram of detomidine HCl monohydrate (sample 1).

FIG. 10 is a TGA thermogram of detomidine HCl monohydrate (sample 1).

FIG. 11 is a XRPD pattern of detomidine HCl monohydrate (sample 2).

FIG. 12 is a DSC thermogram of detomidine HCl monohydrate (sample 2).

FIG. 13 shows a TGA thermogram of anhydrous Detomidine HCl (sample 2).

FIG. 14 is a microphotograph of particle morphology of detomidine HCl monohydrate prepared according to the second procedure (Carbon treatment and detomidine free base isolation, followed by crystallization).

FIG. 15 shows the 1H-NMR spectra of iso-detomidine.

FIG. 16 shows a XRPD pattern of detomidine free base (sample “70”)

FIG. 17 shows a DSC thermogram of detomidine free base.

FIG. 18 shows a TGA thermogram of detomidine free base.

### DETAILED DESCRIPTION OF THE DISCLOSURE

The present invention is based, in part, on the identification of impurities present in detomidine preparations and methods of synthesizing and crystalizing detomidine to reduce the presence of the impurities. The present invention further provides methods to prepare purified solid detomidine HCl for use as a drug substance.

Anhydrous detomidine HCl is available under the brand name, inter alia, Equimidine® and Dormosedan® as a veterinary sedative. Detomidine has not been approved for human use.

In the present disclosure the singular forms “a”, “an” and “the” include the plural reference, and reference to a particular numerical value includes at least that particular value, unless the context clearly indicates otherwise. For example, “the method” includes the broadest definition of the meaning of the phrase, which can be more than one method.

By any range disclosed herein, it is meant that all hundredth, tenth and integer unit amounts within the range are specifically disclosed as part of the invention. Thus, for example, 0.01 mg to 50 mg means that 0.02, 0.03 . . . 0.09; 0.1, 0.2 . . . 0.9; and 1, 2 . . . 49, 50 mg unit amounts are included as embodiments of this invention.

A characteristic of a compound refers to a quality that the compound exhibits, as determined by for example, nuclear magnetic resonance (NMR) spectroscopy (nMS), mass spectroscopy (MS), infrared (IR), ultraviolet (UV) or fluorescence spectrophotometry, gas chromatography (GC), thin layer chromatography (TLC), high performance liquid chromatography (HPLC), elemental analysis, microscopic analysis, Ames test, and includes without limitation peaks, dissolution, stability, crystal shape, particle size, and any other quality that can be determined by an analytical method. Once the characteristics of a compound are known, the information can be used to, for example, screen or test for the presence of the compound in a sample and validate or reject a batch, for example a pharmaceutical batch.

As used herein, “substantially free” refers to a compound or composition having 0.01% area or less of a particular impurity or degradant, or having 0.1% area or less, 0.09% area or less, 0.08% area or less, 0.07% area or less, 0.06% area or less, 0.05% area or less, 0.04% area or less, 0.03% area or less, 0.02% area or less or 0.01% area or less, of total impurities or degradants, relative to detomidine, each as determined by HPLC, using UV detection at 220 nm. In preferred embodiments, the identification of impurities in the human detomidine drug substance is identified by the exemplary HPLC method, as disclosed herein.

As used herein, a “pharmaceutically acceptable” carrier or excipient is one that is suitable for use with humans and/or animals without undue adverse side effects (such as toxicity, irritation, and allergic response) commensurate with a reasonable benefit/risk ratio.

As used herein, “drug substance” refers to the active ingredient in a drug product, which provides pharmacological activity, prior to its incorporation into a drug product, in the treatment or prevention of a symptom or a disease, in a mammal, preferably a human.

As used herein, “drug product” refers to the dosage form containing the drug substance as well as at least one pharmaceutically acceptable carrier or excipient.

As used herein, a “composition” is distinct from a “pharmaceutical composition”, in that it does not include a pharmaceutically acceptable carrier or excipient. A composition as used herein is understood to be present in an inert environment. As used herein, a composition that is “free” of a chemical means that the composition contains, if at all, an amount of the chemical entity which cannot be avoided following an affirmative action intended to eliminate the presence of the chemical in the composition.

As used herein, “about” in the context of a numerical value or range means within ±10% of the numerical value or range recited or claimed.

As used herein, to “treat” or “treating” encompasses, e.g., reducing a symptom, inducing inhibition, regression, or stasis of the disorder and/or disease.

“Administering to the human subject” means the giving of, dispensing of, or application of medicines, drugs, or remedies to a subject/patient to relieve, cure, or reduce the symptoms associated with a condition, e.g., a pathological condition. The administration can be periodic administration.

“Detomidine” refers to the compound, 4-[(2,3-dimethylphenyl)methyl]-1H-imidazole, and to salts and hydrates, thereof. Detomidine is identified by CAS 76631-46-4 and detomidine HCl by CAS 90038-01-0.

Detomidine can be administered in admixture with suitable pharmaceutical diluents, extenders, excipients, or carriers (collectively referred to herein as a pharmaceutically acceptable carrier) suitably selected with respect to the intended form of administration and as consistent with conventional pharmaceutical practices. The unit is preferably in a form suitable for topical administration. Detomidine can be administered alone but is generally mixed with a pharmaceutically acceptable carrier.

General techniques and compositions for making dosage forms useful in the present invention are known to one of skill in the art.

This invention will be better understood by reference to the experimental details which follow, but those skilled in the art will readily appreciate that the specific experiments detailed are only illustrative of the invention as described more fully in the claims which follow thereafter.

### ASPECTS

Aspect 1. Detomidine or a pharmaceutically acceptable salt thereof, that is substantially free of the compound that has a mass-to-charge ratio of 201 and elutes at RRT 0.38 when subjected to high performance liquid chromatography (HPLC);
wherein the HPLC method comprises
   a SunFire C8 column, 100 Å, 3.5 μm, 4.6×150 mm,
   an initial mobile phase that is 70% Ammonium Phosphate buffer solution, pH 7.9, and 30% Acetonitrile,
   a flow rate of 1.0 mL/min, and
   UV detection at 220 nm.

Aspect 2. The detomidine of claim 1, wherein the pharmaceutically acceptable salt of detomidine is detomidine HCl.

Aspect 3. The detomidine of claim 2, wherein the detomidine HCl is the monohydrate form.

Aspect 4. The detomidine of claim 2, wherein the detomidine HCl is the anhydrous form.

Aspect 5. The detomidine any one of claims 1-4, wherein the total amount of impurities is not more than 0.1% area, not more than 0.06% area, or not more than 0.02% area, based on HPLC.

Aspect 6. The detomidine of any one of claims 1-5, wherein the total amount of impurities is not more than 0.06% area, based on HPLC.

Aspect 7. The detomidine of any one of claims 1-6, wherein the detomidine or a pharmaceutically acceptable salt thereof is further substantially free of iso-impurity A (((RS)-(3,4-dimethylphenyl)(1H-imidazol-4-yl)methanol)).

Aspect 8. The detomidine of any one of claims 1-7, wherein the detomidine or a pharmaceutically acceptable salt thereof is further substantially free of impurity A ((RS)-(2,3-dimethylphenyl)(1H-imidazol-4-yl)methanol).

Aspect 9. The detomidine of any one of claims 1-8, wherein the detomidine or a pharmaceutically acceptable salt thereof is further substantially free of impurity B ((RS)-(1-benzyl-1H-imidazol-5-yl)(2,3-dimethylphenyl)methanol) and/or impurity C (4-[(2,3-dimethylcyclohexyl)methyl]-1H-imidazole).

Aspect 10. A composition comprising the detomidine of any one of claims 1-9, and a vehicle.

Aspect 11. A pharmaceutical composition comprising detomidine or a pharmaceutically acceptable salt thereof substantially free of the compound that has a mass-to-charge ratio of 201 and elutes at RRT 0.38 when subjected to high performance liquid chromatography (HPLC); and a pharmaceutically acceptable carrier;
wherein the HPLC method comprises
   a SunFire C8 column, 100 Å, 3.5 μm, 4.6×150 mm,
   an initial mobile phase that is 70% Ammonium Phosphate buffer solution, pH 7.9, and 30% Acetonitrile,
   a flow rate of 1.0 mL/min, and
   UV detection at 220 nm.

Aspect 12. The pharmaceutical composition of claim 11, wherein the pharmaceutically acceptable salt of detomidine is detomidine HCl.

Aspect 13. The pharmaceutical composition of claim 12, wherein the detomidine HCl is the monohydrate form.

Aspect 14. The pharmaceutical composition of claim 12, wherein the detomidine HCl is the anhydrous form.

Aspect 15. The pharmaceutical composition of any one of claims 11-14, wherein the total amount of impurities is not more than 0.1% area, not more than 0.06% area, or not more than 0.02% area, based on HPLC.

Aspect 16. The pharmaceutical composition of any one of claims 11-14, wherein the total amount of impurities is not more than 0.06% area, based on HPLC.

Aspect 17. The pharmaceutical composition of any one of claims 11-16, further substantially free of iso-impurity A (((RS)-(3,4-dimethylphenyl)(1H-imidazol-4-yl)methanol)).

Aspect 18. The pharmaceutical composition of any one of claims 10-17, further substantially free of impurity A ((RS)-(2,3-dimethylphenyl)(1H-imidazol-4-yl)methanol).

Aspect 19. The pharmaceutical composition of any one of claims 10-18, further substantially free of impurity B ((RS)-(1-benzyl-1H-imidazol-5-yl)(2,3-dimethylphenyl)methanol) and/or impurity C (4-[(2,3-dimethylcyclohexyl)methyl]-1H-imidazole).

Aspect 20. A process for the preparation of a monohydrate form of detomidine HCl substantially free of iso-detomidine comprising
a. crystallizing a monohydrate form of detomidine HCl from an aqueous solution of an anhydrous form of detomidine HCl which comprises iso-detomidine; wherein the crystallization takes place by cooling the aqueous solution to a temperature of from about 0° C. to 31° C., optionally in the presence of solid detomidine base or detomidine HCl, until a slurry is formed, and
b. collecting the monohydrate form of detomidine HCl substantially free of iso-detomidine.

Aspect 21. A process for the preparation of a monohydrate form of detomidine HCl substantially free of iso-detomidine comprising
a. treating an aqueous solution of an anhydrous form of detomidine HCl which comprises iso-detomidine with active carbon;
b. converting the detomidine HCl to detomidine base to form an aqueous solution of detomidine base;
c. crystallizing the monohydrate form of detomidine HCl from the aqueous solution of detomidine base by adding a sufficient amount of HCl, wherein the crystallization takes place by cooling the aqueous solution to between about 3° C. and 37° C., optionally in the presence of solid detomidine HCl, until a slurry is formed, and
d. collecting the monohydrate form of detomidine HCl substantially free of iso-detomidine.

Aspect 22. The process of claim 20 or 21, wherein the HCl:detomidine base ratio (mole:mole) is about 1 to about 1.5.

Aspect 23. The process of claim 22, wherein the HCl:detomidine base ratio (mole:mole) is about 1.5.

Aspect 24. The process of any one of claims 20-23, wherein the water:detomidine (V/wt) HCl ratio is about 2-3.

Aspect 25. The process of any one of claims 20-24, wherein the crystallization of the monohydrate form of detomidine HCl from an aqueous solution occurs at a dissolution temperature of between about 35° C.-50° C.

Aspect 26. The process of any one of claims 20-25, wherein the crystallization initiation temperature is about 30° C.-45° C.

Aspect 27. The process of any one of claims 20-26, wherein the monohydrate form of detomidine HCl has a mean crystal size of 0.3 to 0.7 mm.

Aspect 28. The process of claim 27, wherein the shape of the crystals of the monohydrate form of detomidine HCl is rod like and/or prism like.

Aspect 29. The process of any of claims 20-28 wherein the monohydrate form of detomidine HCl has a water content of about 7.5% as determined by Karl Fisher analysis.

Aspect 30. The monohydrate form of detomidine HCl obtained by a process of any one of claims 20-29.

Aspect 31. A composition comprising the monohydrate form of detomidine HCl obtained by a process of any one of claims 20-29; and a vehicle.

Aspect 32. A pharmaceutical composition comprising the monohydrate form of detomidine HCl obtained by a process of any one of claims 20-29; and a pharmaceutically acceptable carrier.

Aspect 33. A method of treating a human subject in need thereof, comprising administering to the human subject the pharmaceutical composition of any one of claims 11-19 and 32.

Aspect 34. The method of claim 33, wherein the human subject is in need of an analgesic.

Aspect 35. A process for validating a batch of a monohydrate form of detomidine HCl drug substance, comprising
a. determining the amount of iso-detomidine in a sample of the batch, and
b. validating the batch for distribution only if the sample of the batch contains not more than 0.01% area of iso-detomidine and/or iso-impurity A relative to detomidine.

Aspect 36. The detomidine of claim 1, which is detomidine free base.

Aspect 37. The detomidine free base of claim 36, having 2 theta values comprising at least one of the values as shown in Table 16, herein.

Aspect 38. Detomidine, or a pharmaceutically acceptable salt thereof, having 0.01% area or less, relative to detomidine, of the compound that has a mass-to-charge ratio of 201 and elutes at RRT 0.38 when subjected to high performance liquid chromatography (HPLC);
wherein the HPLC method comprises
   a SunFire C8 column, 100 Å, 3.5 μm, 4.6×150 mm,
   an initial mobile phase that is 70% Ammonium Phosphate buffer solution, pH 7.9, and 30% Acetonitrile,
   a flow rate of 1.0 mL/min, and
   UV detection at 220 nm.

### EXAMPLES

### Example 1: Elemental Analysis of Impurities Found in Commercially Available Anhydrous Detomidine HCl

### Example 1a

Anhydrous detomidine HCl was sourced from two commercial API suppliers. Properties of the commercial batches, GMP1, GMP2 and GMP3, are presented below.

Elemental impurity analysis was performed by inductively coupled plasma mass spectrometry (ICP-MS) on four different batches of sourced anhydrate. The results of the analysis are found in Table 1.

TABLE 1
Elemental impurities in anhydrous detomidine HCl
Table 1

Batch

| | | | |
|---|---|---|---|
| Non-GMP, | GMP1, | GMP2, | GMP3, |
| Off-white | white | white | white |

| | |
|---|---|
| Elementⁱ⁾ | Content, mg/kg API |

| | | | | |
|---|---|---|---|---|
| Al | 1.4 | <0.5 | <0.5 | <0.5 |
| B | 3.08 | 0.43 | 0.39 | 0.14 |
| Ca | 163 | 28.6 | 27.3 | 19.5 |
| Cr | 0.8 | <0.5 | <0.5 | 0.7 |
| Cu | 1.0 | <0.2 | <0.2 | <0.2 |
| Fe | 5.62 | 0.21 | 0.24 | 0.11 |
| Pd | <0.2 | 0.9 | 3.3 | 5.3 |
| Mg | 5.9 | <1 | <1 | <1 |
| Mn | 2.0 | <0.2 | <0.2 | <0.2 |
| Ni | <0.5 | <0.5 | <0.5 | 0.7 |
| Si | 61.7 | <15 | <15 | <15 |
| S | 201.6 | <15 | <15 | <15 |
| Zn | 17.6 | 1.8 | 2.8 | 1.2 |

ⁱ⁾Elements having levels L.T. 0.5 mg/kg (Ti, As, Hg, Pb, Mo, Pt, etc) are not presented in the table

The screening of elemental impurities shows that the GMP products contained significant levels of Pd (0.9-5.3 mg/kg). Pd is understood to be a catalyst used in the synthesis of detomidine (e.g., in reduction/hydrogenation methods).

### Example 1b: Characterization of Commercially Sourced Material

Samples of the anhydrous detomidine products described in Table 1 were analyzed for water content and characterized by microscope, XRPD and thermal analyses. The results are summarized in Table 2.

TABLE 2
Characterization of commercial anhydrous detomidine HCl

| | Form by | m.p. by DSC | LOD by TGA |
|---|---|---|---|
| Batch | XRPD | ° C. | % |
| Non-GMP, Off- | Mixture^{a} | Two peaks at 95° | 2.7 |
| white | | and 160° | |
| GMP1, white | Mixture^{a} | Two peaks at 95.5° | 0.7 |
| | | and 157° | |
| GMP2, white | Mixture^{a} | A tiny peak at 95° | 0.3 |
| | | and peak at 159.6° | |
| GMP3, white | Mixture^{a} | Two peaks at 95° | 0.6 |
| | | and 159° | |

^{a}Anhydrous + monohydrate

The values presented in Table 2 demonstrate that the commercial samples of detomidine HCl labeled as anhydrous contain some amount of monohydrate and this amount varied depending on storage conditions and packaging.

### Example 2: Stability Assessment of Anhydrate and Monohydrate Forms of Detomidine Base and Detomidine HCl

Pure forms of crystalline free base, and HCl salt (both monohydrate and anhydrate) were prepared from commercially sourced anhydrous detomidine HCl as outlined in Table 3, and characterized using XRPD and thermal analysis. The solids were crystallized from aqueous solutions and then dried under different conditions. The crystallization and drying conditions are summarized in Table 3.

TABLE 3
Preparation of detomidine HCl crystalline forms

| | | Drying conditions | | | |
|---|---|---|---|---|---|
| Sample | | | P; | Time; | Yield |
| No. | Preparation procedure | T ° C. | mbar | hrs | % |
| 1 | Re-crystallization of anhydrous | RT | 20 | 22 | 84.5 |
| | detomidine hydrochloride from 2.88 vol. | | | | |
| | water | | | | |
| 2 | Re-crystallization of anhydrous | 90 | 30-40 | 25 | 84 |
| | detomidine hydrochloride from 2.88 vol. | | | | |
| | water | | | | |
| 3 | Drying under vacuum | 90 | 30-40 | 17 | 100 |
| 4 | Splitting of detomidine hydrochloride | 45 | 30-40 | 13 | 85.2 |
| | with NaOH in water, precipitation and | | | | |
| | isolation of detomidine free base. | | | | |
| | Reaction of detomidine free base with | | | | |
| | HCl 1.05 eq. in 2.80 vol. water | | | | |
| 5 | Carbon treatment of detomidine | 45 | 30-40 | 15.5 | 84.5 |
| | hydrochloride in water, splitting with | | | | |
| | NaOH, precipitation and isolation of | | | | |
| | detomidine free base. Reaction of | | | | |
| | detomidine free base with HCl 1.05 eq. | | | | |
| | in 2.80 vol. water | | | | |

The properties of the solids crystallized according to Table 3 are described in Table 4.

TABLE 4
Properties of Detomidine HCl crystalline forms

| | | m.p. | | LOD | | | |
|---|---|---|---|---|---|---|---|
| | | open | | by | | Water | Particle |
| Sample | | capillary | | TGA | DSC | by cKF | habit, by |
| No. | Color | ° C. | XRPD | % | ° C. | % | microscope |
| 1 | Off- | 158.8-159.8 | Monohydrate | 7.66 | Two melting | 7.36 | Large |
| | white | | | | peaks 94°, | | regular |
| | | | | | 140° | | prisms |
| 2 | Off- | 159.8-160.6 | Anhydrous | 0.04 | Melting at | N.A. | Small |
| | white | | | | 160° | | irregular |
| | | | | | | | particles |
| 3 | Off- | 158.6-160.1 | Anhydrous | 0.3 | Melting at | N.A. | Small |
| | white | | | | 160° | | irregular |
| | | | | | | | particles |
| 4 | Off- | N.A. | Monohydrate | 7.16 | Two melting | N.A. | Large |
| | white | | | | peaks 94°, | | regular |
| | | | | | 157° | | prisms and |
| | | | | | | | rods |
| 5 | White | N.A. | Monohydrate | N.A. | N.A. | 7.46 | Large |
| | | | | | | | regular |
| | | | | | | | prisms and |
| | | | | | | | rods |

These results demonstrate that crystallization from 2.8-2.9 volumes of water is effective for isolation and purification of the detomidine HCl monohydrate drug substance. Drying of the monohydrate under mild conditions (20-40 mbar and temperatures from at least ambient to about 45° C.) provided pure monohydrate without traces of the anhydrous form.

The same monohydrate dried at elevated temperature (30-40 mbar 90° C.) converted completely into the anhydrous form. The vacuum dried, hermetically closed anhydrate did not absorb water from the atmosphere and did not convert into the monohydrate. After exposure to atmospheric air, however, the anhydrate absorbed water and converted to a mixture of anhydrate and monohydrate.

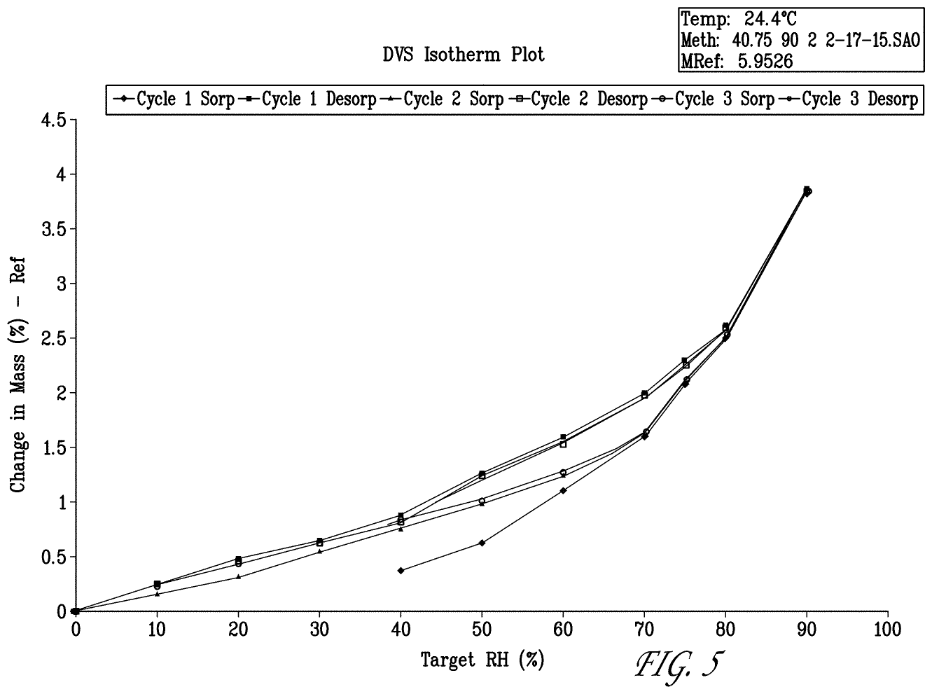

Melting points (m.p.) of the intermediate detomidine free base and hydrochloride of Sample 5 measured in open capillary corresponded with the published literature and the DSC data and are presented in Table 5. In order to evaluate effect of humidity on different forms of detomidine, a hydration study was performed. Samples of detomidine free base and hydrochloride salt were subjected to DVS analysis. These observations are in accordance with the DVS results shown in FIGS. 5 and 6, for detomidine free base and detomidine HCl, respectively.

TABLE 5
Composition and properties of known solid forms of detomidine

| | | | | | m.p., open |
|---|---|---|---|---|---|
| | | Content, % wt. | | | capillary |
| Solid form | MW | Base | HCl | Water | ° C. |
| Free base | 186.2 | 100 | 0 | 0 | 118-119 (114- |
| | | | | | 116)^{a} |
| Hydrochloride | 222.7 | 83.6 | 16.4 | 0 | 158-160 |
| anhydrous | | | | | |
| Hydrochloride | 240.7 | 77.3 | 15.2 | 7.5 | 159-160 (160- |
| monohydrate | | | | | 161)^{a} |

^{a}literature data

The free base was found to be crystalline and insoluble in water but it reacted readily with aqueous HCl giving soluble detomidine hydrochloride.

Crystallization from water provided effective purification of the detomidine HCl and formation of large regular crystals. Anhydrous detomidine hydrochloride appeared as small irregular particles whereas the possibility to control particle size distribution by crystallization parameters existed for the monohydrate.

The detomidine free base was found to be non-hygroscopic, but also able to absorb more than 1% of water at relative humidity (RH)>50%. An increase of humidity from RH 70% to RH>90% did not lead to absorption of additional water to monohydrate. During the dehydration cycle, the monohydrate began to lose water at RH˜10% and converted into the anhydrate at RH=0%. Anhydrate did not absorb water at RH<30% and transformed completely to into the monohydrate at RH between 30% and 50%.

Four cycles of hydration-dehydration demonstrated good reproducibility of anhydrate-monohydrate interconversion.

An anhydrous detomidine HCl of Sample 2 was shown to absorb water to a level of cKF 7.7% which corresponds well to the theoretical amount of water in the monohydrate form (Table 5). The hydration profile of detomidine hydrochloride showed that the monohydrate is stable in a wide range of humidity between 10% and >90% RH. At the same time, the anhydrous form is not stable in atmospheric air and absorbs water at RH=30-50%.

This data demonstrates that the anhydrous form is challenging in the aspects of water content and solid form stability and that detomidine HCl monohydrate is more suitable for pharmaceutical development.

### Example 3: Impurity Analysis of Commercially Sourced Detomidine HCl

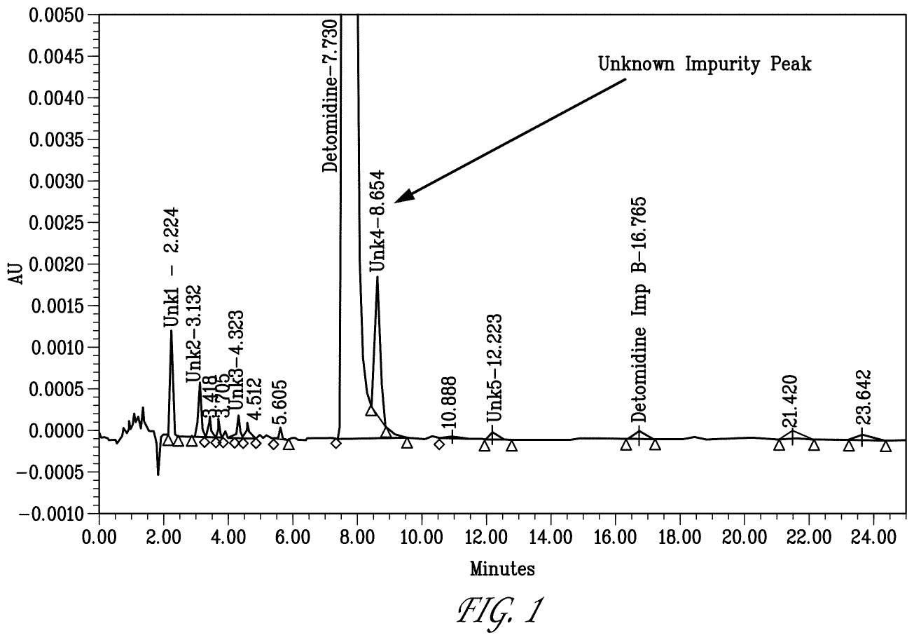

Using the established Pharmacopeia HPLC protocol (Symmetry C8, 5 μm, 4.6×150 mm column, with a mobile phase of 65% Ammonium phosphate buffer pH 7.9 and 35% Acetonitrile at a flow rate of 1.0 mL/min and UV detection at 220 nm), sourced samples of detomidine HCl were assayed for impurities. As shown in FIG. 1, a previously unreported peak was identified, which partially overlapped with that of detomidine. By LC-MS/MS analysis, this impurity was shown to have the same molecular weight as detomidine.

The established Pharmacopeia HPLC protocol did not separate the detomidine from the impurity. Therefore, for further identification of the elusive impurity, new HPLC protocols for assaying detomidine HCl were developed. One protocol (“HPLC Protocol A”) comprised using a SunFire C8 column, 100 Å, 3.5 μm, 4.6×150 mm column with an initial mobile phase of 70% Ammonium Phosphate buffer solution, pH 7.9 and 30% Acetonitrile, at a flow rate of 1.0 mL/min and UV detection at 220 nm. To remove late eluting peaks, the flush gradient shown in Table 6 was applied after each run. This HPLC protocol allowed for a resolution factor of 3.9 between detomidine and the unidentified impurity. The quantitation level (QL) for impurities and degradation products is 0.025%. The detection level (DL) for impurities and degradation products is 0.01%.

TABLE 6
Flush gradient for HPLC protocol

| | % Ammonium Phosphate | |
|---|---|---|
| Time, min | buffer solution | % Acetonitrile |
| 0 | 70 | 30 |
| 25 | 70 | 30 |
| 27 | 30 | 70 |
| 32 | 30 | 70 |
| 33 | 70 | 30 |
| 35 | 70 | 30 |

Given its molecular weight, it was hypothesized that the impurity was iso-detomidine.

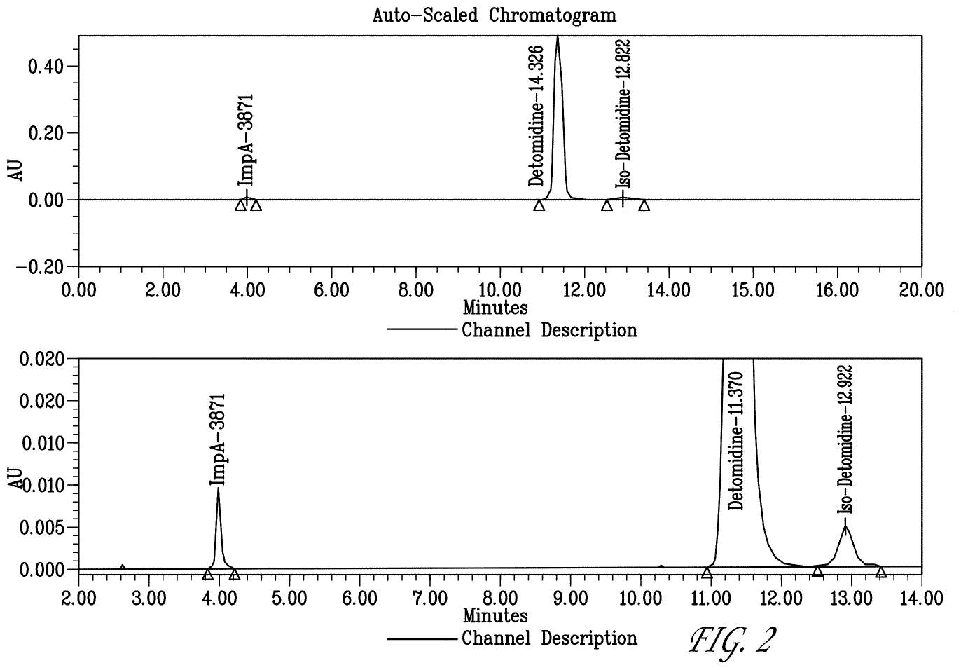

A solution of 100 μg/ml detomidine HCl and about 1 μg/mL (about 1% of the working concentration) of detomidine impurity A and iso-detomidine were prepared and assayed using the new HPLC protocol (HPLC Protocol A), disclosed hereinabove. FIG. 2 is a chromatogram showing that the previously unreported peak is confirmed as being iso-detomidine.

The analysis of commercially sourced detomidine HCl revealed a significant additional impurity. Table 7 provides levels of the various detomidine impurities in different commercial batches. In all batches, total impurities were observed at levels of >0.1% area.

TABLE 7
Impurity levels (% area) in commercial batches of detomidine.

| | | impurity A | | iso- | Total |
|---|---|---|---|---|---|
| Form | Production | (RRT = 0.33) | RRT = 0.38 | detomidine | Impurities |
| Anhydrous | Non-GMP | 0.07 | ND | 0.21 | 0.32 |
| Anhydrous | Non-GMP | 0.15 | ND | 0.02 | 0.19 |
| Monohydrate + | GMP | 0.03 | 0.02 | 0.02 | 0.11 |
| Anhydrous | | | | | |
| (15:85) | | | | | |
| Monohydrate | GMP | 0.04 | 0.17 | 0.01 | 0.3 |
| Monohydrate¹ | GMP | 0.01 | 0.01 | ND | 0.04 |

¹provided by commercial supplier after undergoing the recrystallization process of Example 5, provided by inventors.

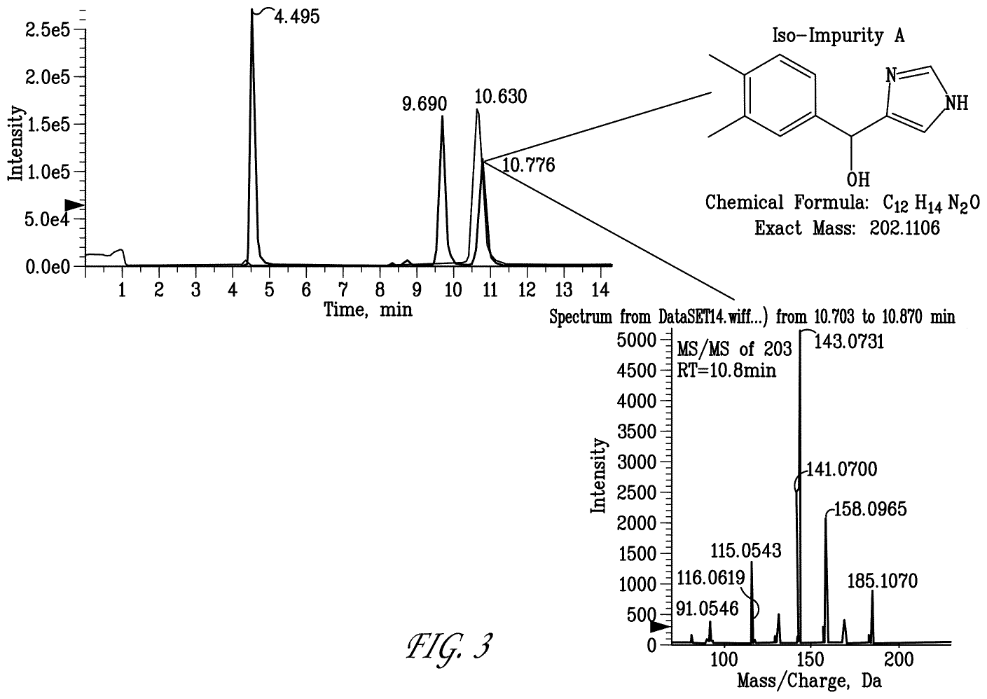

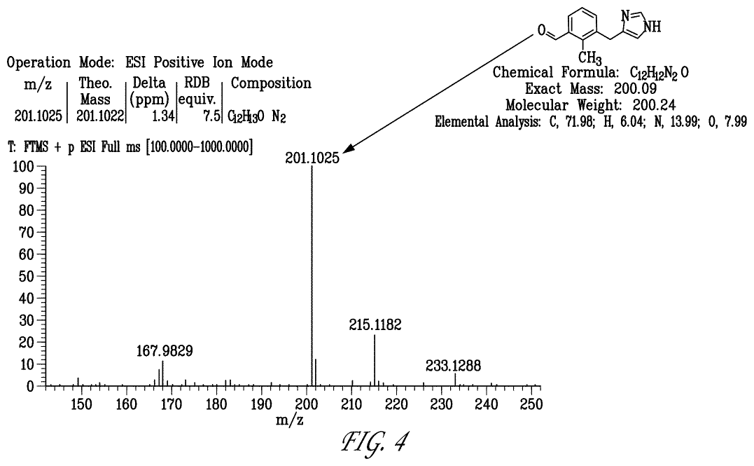

Further analysis of the peak at RRT=0.38 showed that it actually consisted of 2 separate, overlapping peaks. As shown in FIG. 3, LC-MS/MS analysis confirmed one of these peaks as iso-impurity A. After isolation and purification by preparative HPLC, further analysis, using ¹H and ¹³C NMR, LC-MS and HR-ESI-MS and as shown in FIG. 4, the obtained material identified the second peak as 3-((1H-Imidazol-4-yl)methyl)-2-methylbenzaldehyde.

### Example 4: Optimization of the Crystallization Method of Detomidine HCl Monohydrate from Commercial Batches of Anhydrous Detomidine HCl

Crystallization experiments on 25, 65, and 770 gram scale were performed in 100 ml, 500 ml and 3 liter jacketed glass reactors, respectively, equipped with CBT (curved blade turbine) mechanical stirrers, circulating oil bath, thermocouples, and condensers. Stirrer speed in all experiments was between 300-600 rpm. Variable process parameters were: amounts of HCl, solvent ratio, cooling time/rate, seeding and cake wash. The parameters and the variation ranges were chosen according to production conditions. The crystallization parameters are summarized in Table 8.

TABLE 8
Crystallization parameters

| | | | | Temperature | | | | | |
|---|---|---|---|---|---|---|---|---|---|
| | Batch | HCl:base | Water:Det HCl | Solid | Crystallization | End of | Cooling | Cake | |
| | size | ratio | ratio | dissolution | initiation | cooling | time | wash w/ | Yield |
| Batch | gram | Mole:mole | V/wt | ° C. | | | hrs | water | % |
| 82 | 65 | 1.5 | 2.80 | 45 | 38 | 3 | 1.5 | Yes | 91.4 |
| 83 | 65 | 1.5 | 2.80 | 45 | 42^{a} | 3 | 8 | Yes | 93.5 |
| 84 | 25 | 1.0 | 2.80 | Re-slurry of anhydrate:monohydrate | | | | No | 60.8 |
| | | | | mixture (15:85) at 24° C.^{b} | | | | | |
| 85 | 26 | 1.0 | 2.80 | Re-slurry of anhydrate at 24° C.^{b} | | | | No | 65.6 |
| 86 | 26 | 1.5 | 2.80 | 44.6 | 43^{a} | 4 | 4.5 | No | 95.4 |
| 87 | 26 | 1.5 | 2.80 | 46.6 | 43^{c} | 2.5 | 4.5 | No | 95.8 |
| 88 | 26 | 1.5 | 2.80 | 45 | 39^{c} | 3 | 5.5 | No | 94.9 |
| 89 | 26 | 1.0 | 2.10 | 48.7 | 35 | 3 | 5.5 | No | 86.4 |
| 90 | 770 | 1.5 | 2.80 | 46.6 | 45^{a} | 1.5 | 5.5 + 12^{d} | No | 94.6 |
| (sample | | | | | | | | | |
| 3) | | | | | | | | | |
| 91^{e} | 26.5 | 1.0 | 2.80 | 47 | 42^{a} | 3 | 5.5 | Yes | 75.9 |

^{a}Seeding with detomidine HCl monohydrate
^{b}Time 24 hrs
^{c}Seeding with anhydrous detomidine HCl
^{d}5.5 hrs cooling and overnight stirring at 1-3° C.
^{e}Spiked with 2% iso-detomidine

The drying parameters and solid properties of batches shown in Table 8 are described in Table 9.

TABLE 9
Drying parameters and solid properties of detomidine monohydrate crystals

| | Drying | | | | LOD by | H₂O by | PSD | | | | |
|---|---|---|---|---|---|---|---|---|---|---|---|
| | | | Time | XRPD | TGA | CKF | D(0.1) | D(0.5) | D(0.9) | D(1.0) | Crystal |
| Batch | T ° C. | P mbar | Hrs | Formⁱⁱ⁾ | % | % | μm | μm | μm | μm | habit ⁱ⁾ |
| 82 | 30 | 30-40 | 16.5 | M | 6.9 | 7.7 | 174 | 451 | 982 | 2100 | Rods |
| 83 | 30 | 30-40 | 15 | M | 7.3 | 7.9 | 442 | 726 | 1180 | 2100 | Prisms, |
| | | | | | | | | | | | “rounded” |
| 84 | 30 | 20-35 | 20 | M | 8.0 | 7.6 | 173 | 415 | 840 | 1620 | Rods and |
| | | | | | | | | | | | prisms |
| 85 | 30 | 20-35 | 20 | M | 7.7 | 7.3 | 35.9 | 87.3 | 170 | 309 | Irregular |
| | | | | | | | | | | | aggregated |
| | | | | | | | | | | | particles |
| 86 | 30 | 25-28 | 19 | M | 7.7 | 7.4 | 296 | 536 | 842 | 1260 | Rods and |
| | | | | | | | | | | | prisms |
| 87 | 30 | 25-28 | 19 | M | 7.6 | 7.2 | 193 | 340 | 551 | 859 | Clusters of |
| | | | | | | | | | | | small |
| | | | | | | | | | | | prisms |
| 88 | 30 | 25-30 | 17 | M | 7.7 | 7.4 | 133 | 287 | 438 | 661 | Rods |
| 89 | 30 | 25-30 | 17 | M | 7.7 | 7.5 | 284 | 556 | 961 | 1630 | Rods and |
| | | | | | | | | | | | prisms |
| 90 | 30-40 | 18-35 | 20 | M | 7.6 | 7.4 | 246 | 434 | 645 | 898 | Prisms, |
| | | | | | | | | | | | “rounded” |
| 91 | 35 | 18-21 | 16 | N.A. | N.A. | N.A. | 423 | 743 | 1270 | 2380 | Prisms |

ⁱ⁾ microscopic observation: Rods—aspect ratio > 2; prisms—aspect ratio < 2
ⁱⁱ⁾M = monohydrate

The data presented in Tables 8 and 9 demonstrate that crystallization from water and drying under technical vacuum gives pure detomidine HCl monohydrate without traces of the detomidine HCl anhydrous form. Variations of HCl excess from 0 to 0.5 mole/mole base, cooling time from 1.5 to 24 hours and drying time from 15 to 33 hours appear to have no effect on the obtained properties of the solid form. All crystallization products appeared as pure detomidine HCl monohydrate.

The crystallization initiation method also had no effect on crystalline form. The batches seeded with anhydrous material gave the same monohydrate as batches seeded with monohydrate and batches which crystallized spontaneously.

Contact with water for 24 hrs completely converted the anhydrous form into the monohydrate, even without complete dissolution (re-slurry).

Crystallization of the monohydrate from water gave large clear crystalline particles with a mean crystal size 0.3-0.7 mm, with some crystals larger than 2 mm in size. The shape of the crystals was rod-like or prism-like, if the aspect ratio of the crystals was <2 the crystals were reported in Table 8 as prisms.

A ratio of HCl to base within the range 1.0-1.5 mole:mole and water to solid ratio within the range 2.1-2.8 V/wt were found to have no significant effect on the particle size distribution (PSD). However, a ratio of HCl to base of about 1.5 were found to increase yields of highly pure detomidine HCl monohydrate from under 90% (60.8%-86.4%) to over 90% (91.4%-95.9%). Seeding also appeared to have no significant effect on PSD.

The cooling rate was found to have a weak effect on PSD. There was no effect observed for cooling over a time range between 1.5 and 5.5 hrs (mean cooling rate 0.10-0.31° C./min).

Slurry-to-slurry recrystallization of anhydrous material resulted in a strong reduction in particle size with the d(0.5) decreasing from 300-500μ to 87μ. These crystals were found irregular with no signs of prism-like or rod-like habit. In contrast, the re-slurry procedure applied to a mixture of anhydrate and monohydrate (15:85) gave a mixture of rod and prism-like crystals with d(0.5)=415μ.

Batch size was found to have no significant effect on crystal size and shape. After scaling up from a 26 g batch in 100 ml reactor to 770 g in a 3 liter reactor, the PSD was very similar to that of small scale batches.

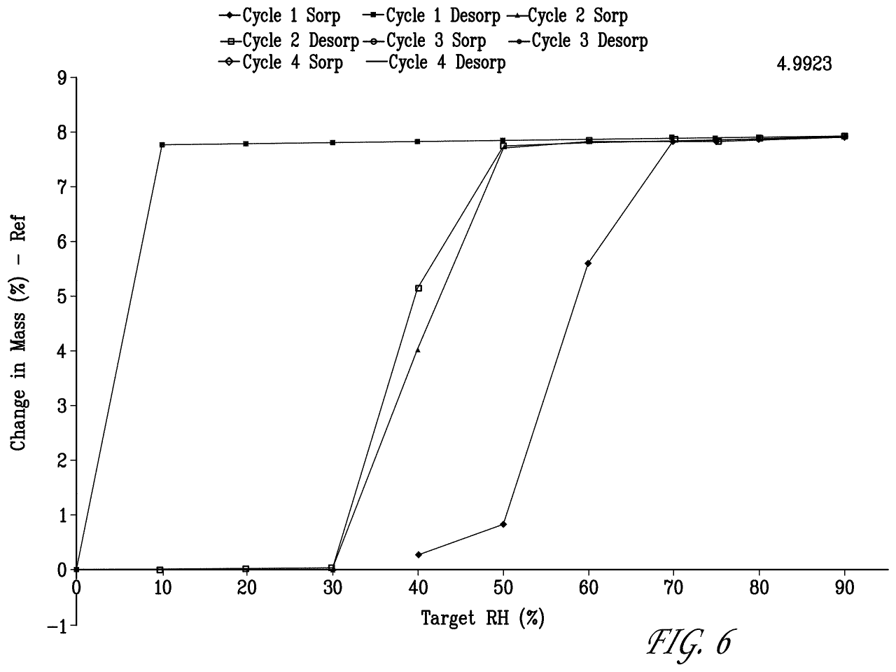

Prolonged cooling resulted in a “rounded” form of crystals. This effect was observed in two experiments, as seen in the microscopic photograph in FIG. 7. In the first experiment the crystallizing suspension was cooled for 8 hrs, and in the second one it was stirred at low temperature for 12 hrs (batches 83 and 90 in Tables 8 and 9).

Under the conditions described, cooling had a strong effect on the process yield. Two re-slurry experiments were performed at the same water volume ratio as most of experiments (2.80 V/wt) but these two batches were not cooled and filtered at 24° C. In these experiments the yield dropped from 86% to 60-65% (batches 84, 85 in Tables 8 and 9).

Acceptable yields were obtained in cooled batches within the solvent volume ratio range 2.1-2.8 V/wt with the cooling temperature between about 1.5° C.-4° C.

An increase of HCl to base molar ratio from 1 to 1.5 was found to raise the yield from 86% to 95%. Cake wash reduced the yield by 2-3%. Re-crystallization in presence of 2% iso-detomidine reduced the yield from 84-85% to 76%. The purity of the samples prepared according to methods disclosed in Tables 8 and 9, determined using the optimized HPLC method, are presented in Table 10.

TABLE 10

| | | After crystallization | | | | | | |
|---|---|---|---|---|---|---|---|---|
| | Before | Batch | | | | | | |
| | crystallization | 83 | 84 | 85 | 86 | 87 | 88 | 89 |
| Color | Non GMP, | White | White | White | White | White | White | White |
| | White | | | | | | | |

Impurities by HPLC, % area

| | | | | | | | | |
|---|---|---|---|---|---|---|---|---|
| RRT = 0.20- | ND | ND | ND | ND | ND | ND | ND | ND |
| 0.21% area | | | | | | | | |
| Impurity A | 0.15 | 0.03 | 0.04 | 0.05 | 0.04 | 0.04 | 0.04 | 0.04 |
| (RRT = 0.33% | | | | | | | | |
| area) | | | | | | | | |
| RRT = 0.43% | 0.01 | ND | ND | ND | ND | ND | ND | ND |
| area | | | | | | | | |
| RRT = 0.61% | ND | 0.01 | ND | ND | ND | ND | ND | ND |
| area | | | | | | | | |
| iso-detomidine | 0.02 | ND | ND | ND | ND | ND | ND | ND |
| (RRT = 1.14% | | | | | | | | |
| area) | | | | | | | | |
| Total Impurities | 0.19 | 0.04 | 0.04 | 0.05 | 0.04 | 0.04 | 0.04 | 0.04 |

### Example 5: Purification of Organic Impurities from Detomidine HCl Monohydrate

Two potential procedures for purification of organic impurities from sourced monohydrate were compared. The first attempted procedure was by direct re-crystallization of detomidine HCl from 2.88 volumes of water, while the second included carbon treatment and precipitation of detomidine free base followed by the free base being reacted with HCl and crystallized as monohydrate. Both procedures used the same non-GMP, off white anhydrous detomidine HCl starting material which had previously been shown in Table 7 to contain 0.21% of iso-detomidine and 0.07% of Impurity A. All the re-crystallized materials were found to have practically the same purity level. The direct re-crystallization procedure was found to provide a product with a high yield and purity and at the same time provides a practical and scalable crystallization process which could be controlled by process parameters such as seeding and cooling rate.

### Example 5a: Direct Recrystallization

Anhydrous detomidine HCl (4.5 g) was introduced to a round-bottom flask with a magnetic stirrer and thermometer. Deionized water (13 ml) was then added and the mixture stirred and heated in a water bath. At 39° C., the complete dissolution of solids was observed, providing a clear yellow solution with a pH=4.

The batch was gradually cooled by stirring. At 31° C., intensive crystallization was observed. The resulting slurry was cooled in an ice-water bath for 20 min and filtered. Flask and cake were then washed with 2 ml of cold deionized water and 3.97 g of a white to cream colored solid was collected.

2.03 g of the material was dried in a vacuum desiccator at ambient temperature and 20 mbar to a constant weight over 23 hrs producing a dry monohydrate −1.96 g off-white crystalline solid (sample 1).

An additional 1.91 g of the material was dried in a vacuum oven at 90° C. under house vacuum to a constant weight over about 24.5 hrs producing a dry anhydrate, 1.68 g off-white solid (sample 2)

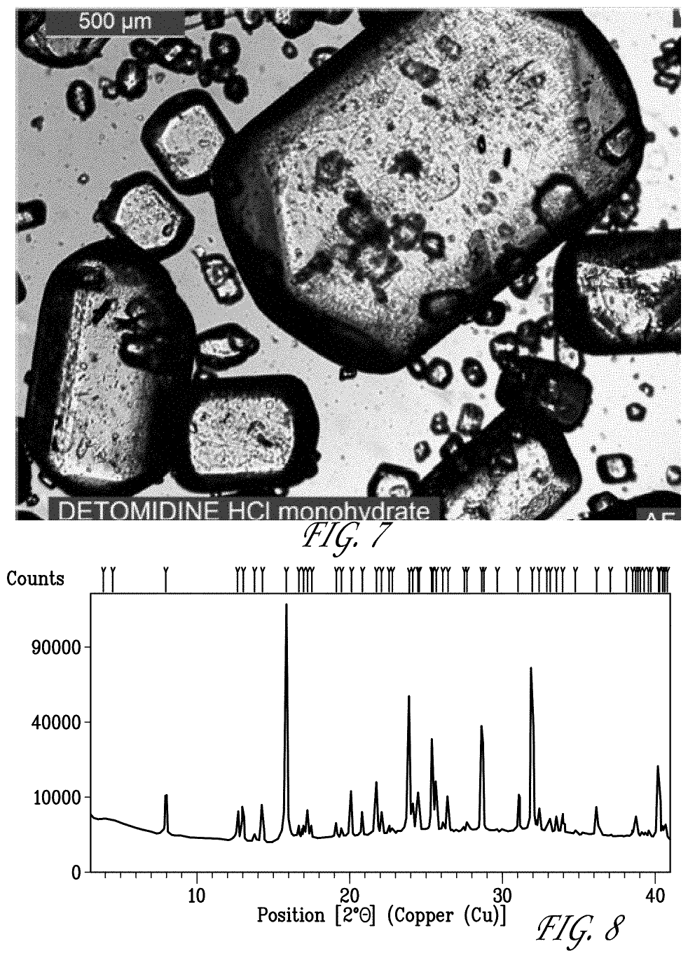

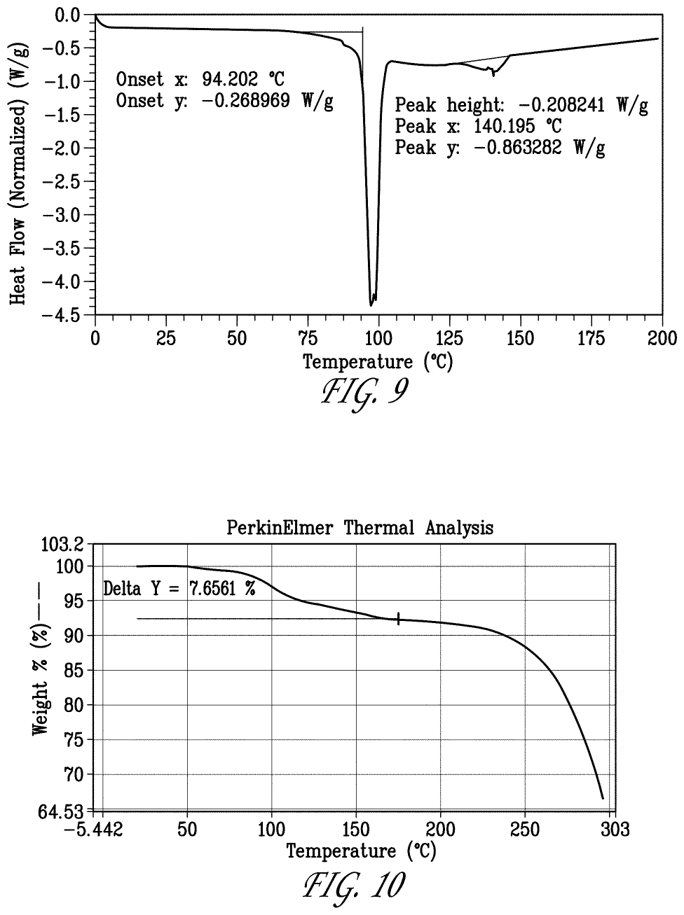

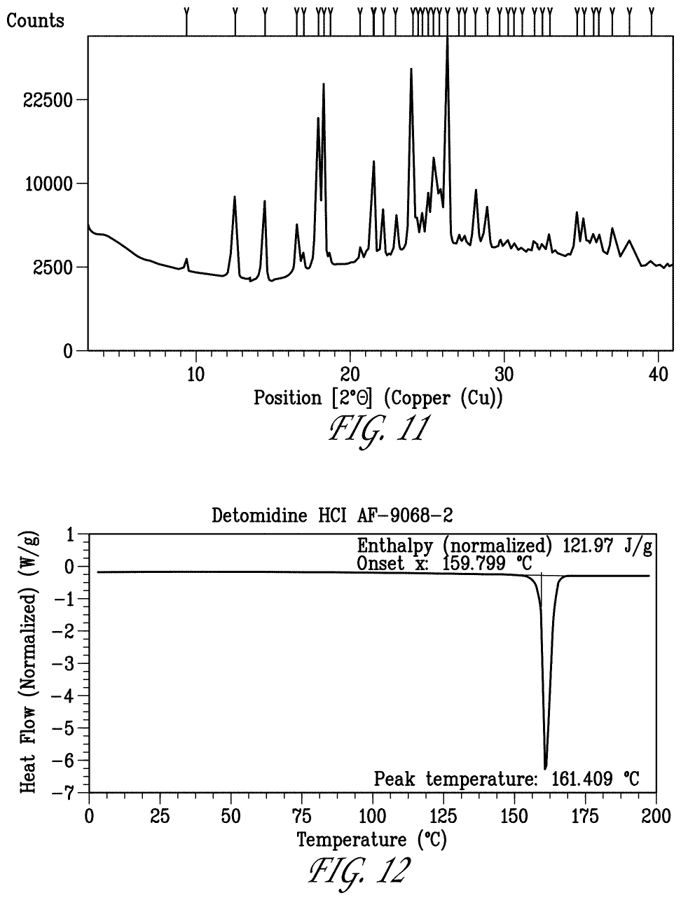

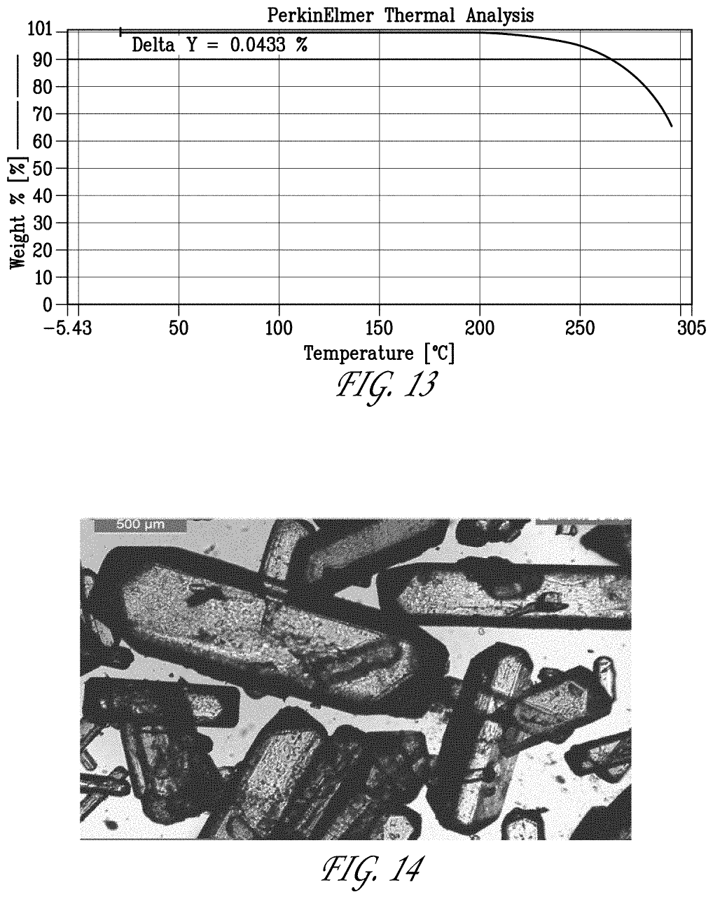

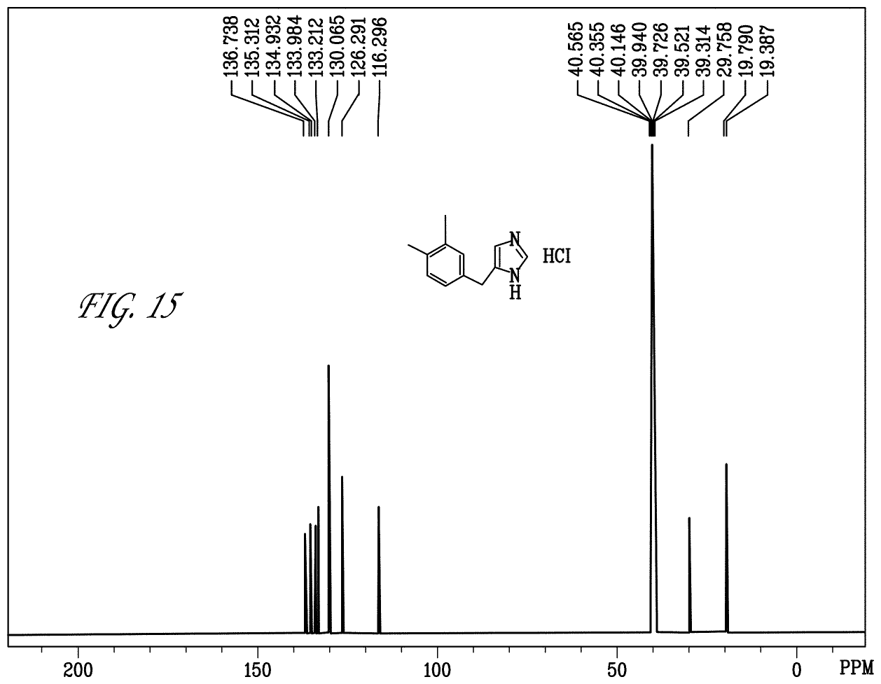

The two samples were subjected to physical characterization and purity analysis by HPLC. The XRPD spectra and DSC and TGA thermograms of sample 1 are presented in FIGS. 8-10 and of sample 2 are presented in FIGS. 11-13, respectively.

As shown in Table 11, direct re-crystallization resulted in the effective purification from all organic impurities, but was not effective for color. The content of iso-detomidine and of Impurity A was reduced to a level below the QL, but the off white color remained after re-crystallization.

TABLE 11
properties following direct recrystallization (sample 1)

| | Before | After |
|---|---|---|
| | re-crystallization | re-crystallization |
| Color | Off-white | Off-white |

Impurities by HPLC, % area

| | | |
|---|---|---|
| RRT = 0.20-0.21% area | 0.06 | 0.02 |
| Impurity A (RRT = 0.33) | 0.07 | 0.01¹ |
| RRT = 0.48% area² | 0.01 | ND |
| iso-detomidine (RRT = 1.14) | 0.21 | 0.01¹ |
| Total Impurities | 0.32 | 0.02 |

¹below the QL
²system peak

### Example 5b(i): Carbon Treatment and Detomidine Free Base Isolation

Anhydrous detomidine HCl (70.3 g) and deionized water (220 ml) were introduced to a 0.5 liter jacketed glass reactor equipped with a mechanical stirrer, thermocoupler and a circulating oil bath for heating and cooling.

The mixture was heated while stirring. At 40° C., complete dissolution was observed. Active carbon (CXV type, 5.2 g) was added to the clear yellow solution and the batch stirred at 45° C. for 50 minutes. Following this, the batch was filtered on through paper filter on Büchner funnel, reactor and filter washed with deionized water (20 ml).

The slightly yellowish clear filtrate was reintroduced to the 0.5 liter reactor, stirred and 40% NaOH solution was added at 40° C. After 10 ml NaOH solution was added, a pH of 7 was reached and precipitation began. An additional 13 ml of NaOH was added over 1 hour at 42-52° C. and intensive stirring (400-450 rpm) performed. The mixture at the end of the addition of NaOH had a pH of 13.

The batch was stirred at 33-35° C. overnight then cooled to 16° C. over 4 hours and stirred at this temperature for an additional hour. The resultant solid was filtered on Büchner filter, reactor and cake washed with two portions of deionized water (2×200 ml). The wet solid (86 g) was dried in a vacuum oven at 45° C. to constant weight to produce a dry product (53.2 g, Yield 90.7%)—white powder, m.p.=118.6-119.2

The dry detomidine base was analyzed for purity by HPLC, the results presented in Table 12.

TABLE 12
Properties of detomidine base (intermediate in sample 2)

| | Before | Free base |
|---|---|---|
| | purification | (dry) |
| Color | Off-white | White |

Impurities by HPLC, % area

| | | |
|---|---|---|
| RRT = 0.20-0.21% area | 0.06 | 0.05 |
| Impurity A (RRT = 0.33) | 0.07 | 0.02 |
| RRT = 0.48% area¹ | 0.01 | 0.01 |
| iso-detomidine | 0.21 | 0.13 |
| (RRT = 1.14% area) | | |
| Total Impurities | 0.31 | 0.22 |

¹system peak

### Example 5b(ii): Monohydrate Crystallization from Detomidine Base

The dry detomidine free base (53.0 g) from Example 5b(i) was introduced together with 37% HCl (29.7 g) and deionized water (159 g) into a 0.5 liter jacketed glass reactor equipped with a mechanical stirrer, a thermocoupler and a circulating oil bath for heating and cooling. The batch was stirred and heated to 45° C., at 37° C. complete dissolution of solid was observed. The clear solution had a pH of 1. The solution was cooled gradually to 37° C. and seeded with detomidine HCl monohydrate and cooled gradually to 3° C. over 4 hours, and then the batch was stirred for 45 minutes at this temperature.

The solid was filtered on Büchner filter, reactor and cake washed with cold deionized water (80 ml). The wet solid (61.9 g) was dried in vacuum oven for 16 hours at 45° C. to produce a dry product (57.8 g, Yield 84.3%)—white crystalline powder (sample 2)

The dry detomidine HCl monohydrate was analyzed for water by CKF (H₂O=7.46%) and for purity by HPLC with the results presented in Table 13. Microscopic observation for particle morphology (regular prisms) was performed and the microscopic photograph is shown in FIG. 14.

TABLE 13
Properties of detomidine HCl (sample 2)

| | Before | Monohydrate after |
|---|---|---|
| | purification | crystallization |
| Color | Off-white | White |

Impurities by HPLC, % area

| | | |
|---|---|---|
| RRT = 0.20-0.21% area | 0.06 | ND |
| Impurity A (RRT = 0.33% area) | 0.07 | ND |
| RRT = 0.48% area¹ | 0.01 | ND |
| iso-detomidine (RRT = 1.14% | 0.21 | ND |
| area) | | |
| Total Impurities | 0.31 | ND |

¹system peak

### Example 5c: Re-Crystallization of Detomidine HCl to Monohydrate, Bench Scale Experiment

Anhydrous detomidine HCl (754.6 g) 37% HCl (116.0 g) and deionized water (2008 g) were introduced to a 3 liter glass jacketed reactor equipped with a mechanical stirrer, two baffles, a thermocoupler and a circulating oil bath for heating and cooling. The batch was stirred and heated to 52° C., at 47° C. complete dissolution was observed and the clear solution was found to have a pH of 0-0.5.

The solution was cooled gradually and at 45° C. seeded with detomidine HCl monohydrate (0.5 g). Crystallization initiation was observed at 43° C. and the batch was then cooled to 1.5° C. during 5 hours and stirred for 12 hours at this temperature. The solid was filtered on Büchner filter and conditioned on the filter with vacuum for 40 minutes. The wet product (817 g) was dried in vacuum oven to constant weight.

For the first 13 hours, the material was dried at 30° C. and 35-27 mbar, then for an additional 7 hours at 40° C. and 30-18 mbar to produce a dry product (771.2 g, Yield 94.6%)—white crystalline powder (Batch “90” in Tables 8-9; sample 3)

Dry detomidine HCl monohydrate was analyzed for water by CKF (H₂O=7.37%) and for purity by HPLC, the results presented in Table 14. The physical characterization results are shown in Table 10 above.

The material was subjected to physical characterization and microscopic observation for particle morphology (regular prisms) microscopic photograph presented in FIG. 7.

TABLE 14
Properties of detomidine HCl (sample 3)

| | Before | Monohydrate after |
|---|---|---|
| | re-crystallization | crystallization |
| Color | Non GMP, white | White |

Impurities by HPLC, % area

| | | |
|---|---|---|
| RRT = 0.20-0.21% area | ND | 0.03 |
| Impurity A (RRT = 0.33% area) | 0.15 | 0.03 |
| KKT = 0.48¹% area | 0.01 | ND |
| RRT = 0.61¹% area | ND | ND |
| iso-detomidine (RRT = 1.14% | 0.02 | ND |
| area) | | |
| Total Impurities | 0.19 | 0.06 |

¹system peak

### Example 6: Synthesis of Iso-Detomidine

Scheme 1 outlines a process for the synthesis of iso-detomidine was developed to produce a solid iso-detomidine HCl in high yield and substantially free of impurities.

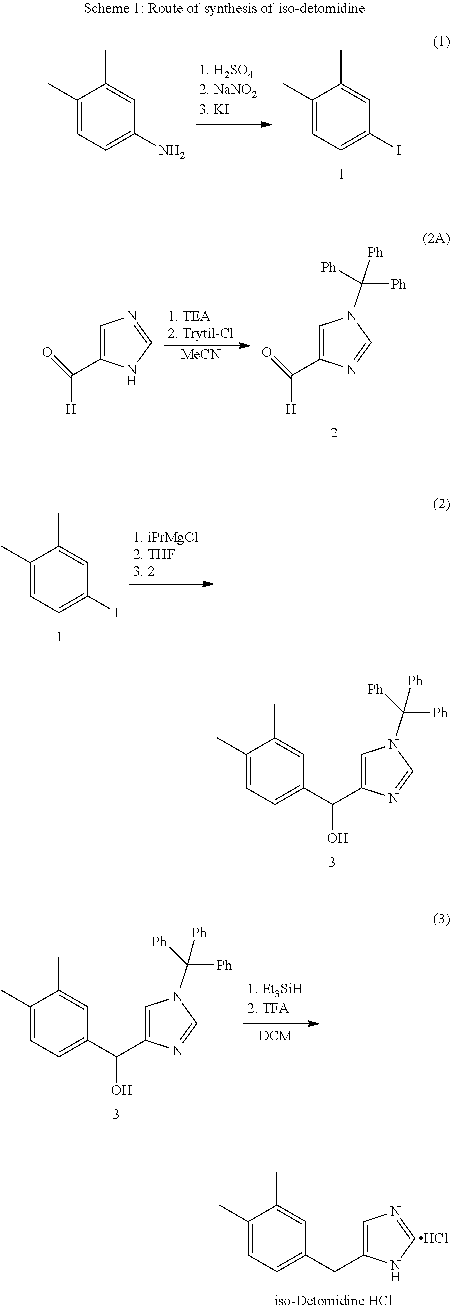

### Example 6a: Sandmeyer Reaction

3,4 dimethyl aniline (150 g, 1.24M) was mixed with acetonitrile (0.6 liter) in a 5 liter flask, chilled to 10° C. and water (1.2 liter) added dropwise over 5 minutes. The mixture was cooled to 5° C. with ice-ethanol bath and concentrated H₂SO₄ (98% wt., 363 g 3.71M) was added dropwise over 30 min at 5-10° C.

Sodium nitrite (NaNO₂) aqueous solution (89.7 g in 300 ml water, 1.30M) was then added dropwise over 30 min at 0-5° C. to give a brown solution. The resulting solution of diazonium salt was stirred at 0-5° C. for an additional 30 min.

In another 5 liter flask KI (225 g, 1.36M) was dissolved in water (0.8 liter) during stirring and cooled. The diazonium salt solution was added dropwise to the KI solution at 7-13° C. during 35 min, the batch stirred at 7-13° C. for 1.25 hr to give a black solution. MTBE (2.0 liter) was then added to the reaction mixture and Na₂SO₄ (23.4 g) was introduced in small portions during 5 min.

The mixture was settled and the organic phase separated and washed with two portions of brine (2×500 ml). The organic solution was concentrated under vacuum to a volume of about 250 ml. The product was purified by vacuum distillation at ca. 40 Pa, BP=52-60° C. to give 246 g of intermediate 1 as a brown oil with a product yield of 86%.

### Example 6b: TRT Protection Reaction

1H-Imidazole-4-carbaldehyde (45.2 g, 0.47M) and acetonitrile (0.8 liter) are introduced into a 2 liter flack and cooled to 8° C., then TRT-Cl (131.0 g, 0.47M) was added at 8° C. and TEA (57.1 g, 0.56M) was added dropwise during 20 min. The reaction mixture was stirred at 8 to 18° C. for 2 hrs.

The reaction mixture was poured into a stirring mixture of water (0.72 liter) and MTBE (0.72 liter) and stirred for 10 minutes. The resulting solid was isolated by filtration on Büchner funnel and dissolved with THF (3 liter). The solution was dried over Na₂SO₄ and concentrated to remove most of the solvent.

MTBE (400 ml) and PE (200 ml) was added to the residue, the mixture stirred at 8° C. for 16 hrs. The precipitated solid was isolated by filtration on Büchner filter and dried in air for 16 hrs at room temperature. Then the filter cake is dried by azeotropic drying with 2-Me-THF (2×500 ml) to give 129 g of intermediate 2 as white solid with a yield of 66.5%.

### Example 6c: Grignard Reaction

A 2M solution of i-PrMgCl in THF (0.275 liter, 0.55M) and THF (1.0 liter) was introduced to a 2 liter flask at 12° C. Intermediate 1 (121.8 g, 0.525M) was added dropwise during 20 min. The mixture was stirred at 12-15° C. for 3 hrs.

Intermediate 2 (84.6 g, 0.25M) was added in small portions without cooling during 30 min, with a temperature rise to 25° C., to give a light brown solution. The solution was stirred for 2.5 hrs at 15° C. and added to aqueous solution of NH₄Cl (117 g in 0.7 liter water) during 10 min at 5° C. PE (1.6 liter) was added during 5 min and the mixture stirred for extra 25 min.

Precipitated solid filtered on Büchner funnel and then re-slurred with mixture of MTBE (400 ml), water (600 ml) and PE (200 ml). Then the solid was filtered on Büchner funnel and re-slurred with MeOH (700 ml) at 60° C. for 10 min, cooled to 20° C. with cold water bath and filtered again on Büchner funnel. The solid product was dried in an air oven at 45° C. for 2 hrs to give 112 g of intermediate 3 as a white solid with a yield of 89.9%.

### Example 6d: Reductive Dehydroxylation and De-Protection

Intermediate 3 (107 g, 0.240M) and DCM (1.10 liter) were introduced to a 2 liter flask at 11° C., TFA (214 ml) was added dropwise over 5 mins with a temperature rise to 14° C.

The mixture was stirred for about 5 mins and Et₃SiH (94.4 g, 0.794M) added dropwise during 5 mins. After stirring at 25-30° C. for 16 hrs the mixture was concentrated by rotary evaporation at 40° C. to a residue.

The residue of evaporation was dissolved in DCM (600 ml) and washed with 1.5M aq. HCl (0.24 liter). Organic phase was separated and washed with aq. NaOH (11.5 g in 200 ml water), pH of aqueous phase 13. Two phases were separated and the organic phase washed with brine (200 ml) dried over Na₂SO₄ and filtered. The resulting solution was concentrated by rotary evaporation.

The evaporation residue was dissolved in mixture of EtOAc (500 ml) and EtOH (30 ml) and then 4M HCl solution in dioxane (40 ml) was added dropwise in 5 minutes, pH=1-2 adjusted and a white solid precipitated out.

The solid product was filtered on Büchner funnel, the cake dried in air for 16 hrs to give 36 g of white solid.

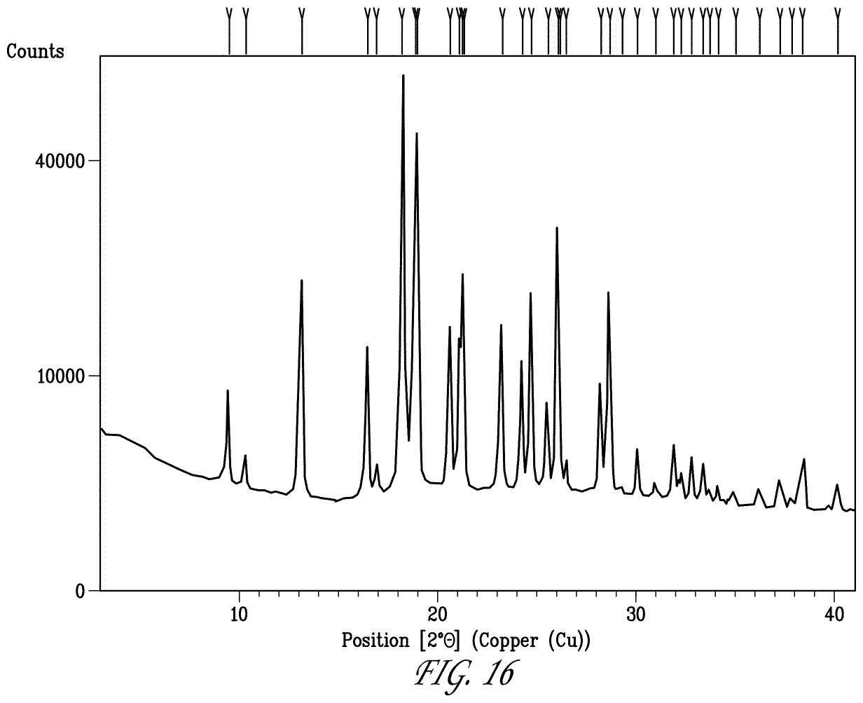

The solid product was re-crystallized from iPrOH/Acetone. The dry cake (36 g) and iPrOH were introduced into a 1 liter flask and heated to dissolution. Acetone (360 ml) was added to the resulting colorless solution at reflux during 10 mins. The mixture was cooled to 8° C. and stirred at this temperature for additional 4.5 hrs. The solid product was filtered on Büchner funnel and dried in air for 36 hrs. 29.2 g of iso-detomidine as a white solid was obtained with a yield of 54.4%. The ¹H-NMR spectra of iso-detomidine is shown in FIG. 15.

### Example 7: Re-Crystallization of Detomidine HCl Spiked with 2% Iso-Detomidine

Detomidine HCl monohydrate (26.0 g), iso-detomidine HCl (0.52 g) and deionized water (68.7 g) were introduced to a 100 ml glass jacketed reactor equipped with a mechanical stirrer, a thermocouple and a circulating oil bath for heating and cooling. The batch was stirred and heated to 51° C., at 47° C. complete dissolution was observed.

The solution was cooled gradually and at 42° C. seeded with detomidine HCl monohydrate. Crystallization initiation was observed at 39° C. and then the batch was cooled to 3° C. for 5 hours, filtered on Büchner filter and conditioned on the filter with vacuum. The wet product (20.7 g) was dried in vacuum oven to constant weight to produce a dry product (20.13 g, Yield 75.9%)—white crystalline powder

Dry detomidine HCl monohydrate was analyzed for PSD and morphology, the results are presented in Table 8 (Sample. No. 91). The purity of re-crystallized material was analyzed using the optimized HPLC process disclosed herein, and the results are presented in Table 15.

TABLE 15
Properties of detomidine HCl following recrystallization
from iso-detomidine spiked material

| | Before | After |
|---|---|---|
| | purification | purification |
| Color | White | White |

Impurities by HPLC, % area

| | | |
|---|---|---|
| Impurity A (RRT = 0.33)^{a} | 0.05 | 0.01 |
| RRT = 0.46-0.48 | ND | 0.03 |
| iso-detomidine HCl | 2.17^{b} | 0.08 |
| (RRT = 1.14) | | |
| Total Impurities | 2.23 | 0.11 |

^{a}area %
^{b}Spiked amount, calculated

### Example 8: Properties of Solid Form Detomidine Base

### Example 8a: X-Ray Powder Diffraction (XRPD) Kα1Mode

XRPD patterns were recorded on a PANalytical X Pert Pro diffractometer equipped with an Xcelerator detector using Cu Kα radiation at 45 kV and 40 mA.

Kα1 radiation was obtained with a highly oriented crystal (Ge111) incident beam monochromator. A 10 mm beam mask, and divergence)(¼°, and anti-scatter)(⅛° slits were inserted on the incident beam side. Receiving (5 mm) and Soller (0.04 rad.) slits were inserted on the diffracted beam side. The XRPD scan was collected from ca. 2 to 40° 2θ with a 0.0080° step size and 96.06 sec counting time which resulted in a scan rate of approximately 0.5°/min. The sample was spread on a glass plate or a silicon zero background (ZBG) plate for the measurement. The sample was rotated at 15 revolutions/min on a PANalytical PW3065/12 Spinner. Measurement of the Si reference standard before the data collection resulted in values for 20 and intensity that were well within the tolerances of 28.38<20<28.50 and significantly greater than the minimum peak height of 150 cps.

### Example 8b: XRPD Standard Mode

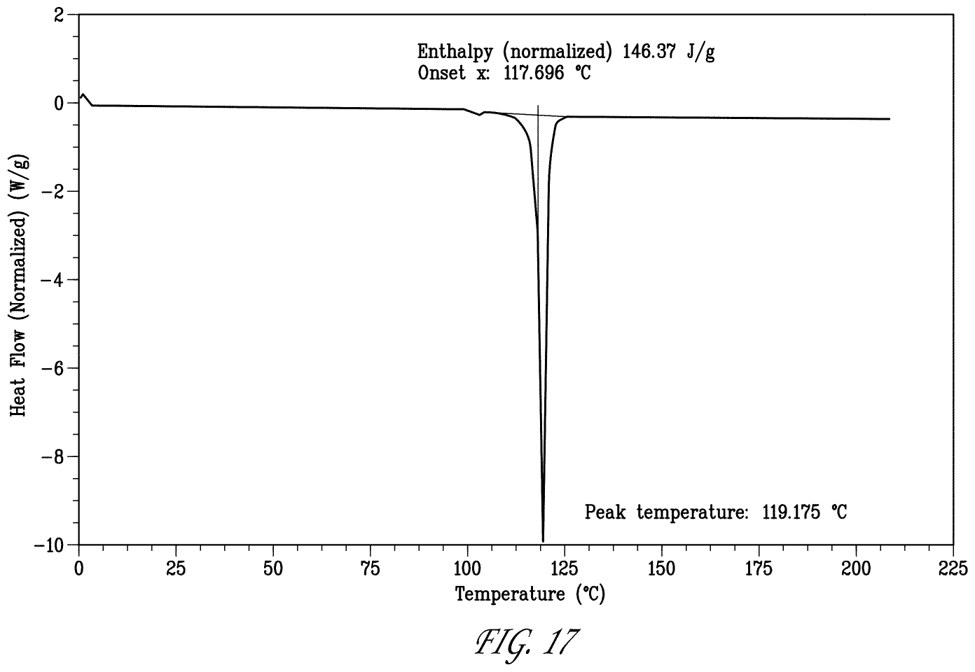

XRPD patterns were recorded on a PANalytical X Pert Pro diffractometer equipped with an X celerator detector using Ni filtered Cu Kα radiation at 45 kV and 40 mA. A 10 mm beam mask, and divergence)(¼° and anti-scatter (⅛°), and Soller (0.04 rad.) slits were inserted on the incident beam side. Receiving (5 mm) and Soller (0.04 rad.) slits and a Ni filter were inserted on the diffracted beam side. The X-ray powder pattern scan was collected from ca. 2 to 40° 2θ with a 0.0080° step size and 96.06 sec counting time which resulted in a scan rate of approximately 0.5°/min. The sample was spread on a glass plate or a silicon zero background (ZBG) plate for the measurement. The sample was rotated at 15 revolutions/min on a PANalytical PW3065/12 Spinner. Measurement of the Si reference standard before the data collection resulted in values for 20 and intensity that were well within the tolerances of 28.38<2θ<28.50 and significantly greater than the minimum peak height of 1000 cps. The XRPD pattern of detomidine free base is shown in FIG. 16.

### Example 8c: DSC—Differential Scanning Calorimetry

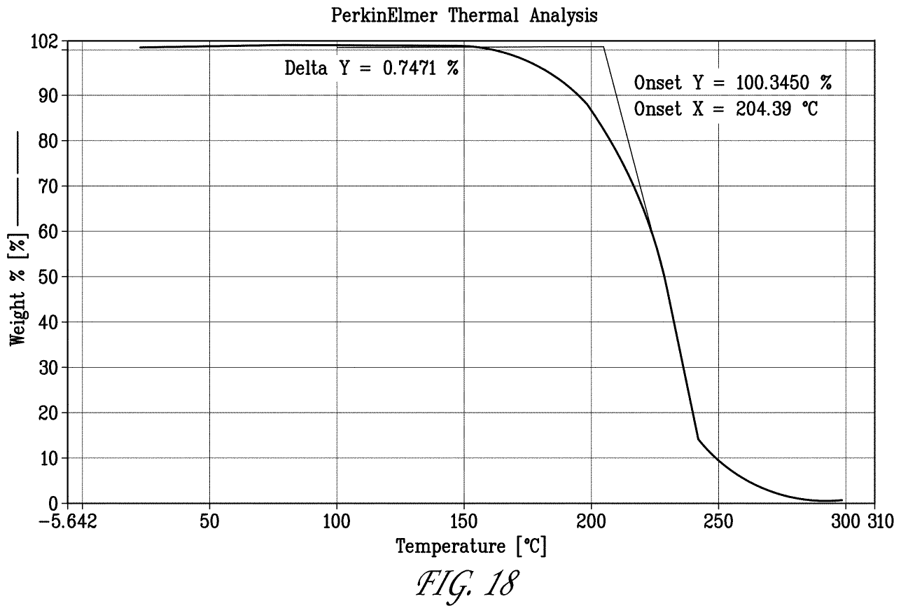

Thermal curves were acquired using a TA Discovery DSC unit. Solid samples of 5-20 mg were weighed into Tzero™ aluminum pinhole hermetically sealed pin hole pans. The DSC cell was then purged with nitrogen and the temperature heated at 10° C./min. from 0° to 200° C. Indium (Tm=156.6° C.; ΔHFus=28.45 J g-1) was used for calibration. The DSC thermogram of detomidine free base is shown in FIG. 17.

### Example 8d: TGA—Thermogravimetric Analyzer

Thermal curves were acquired using a Perkin-Elmer Pyris 1 TGA unit running Pyris software version 6.0 calibrated with alumel (95% nickel, 2% manganese, 2% aluminum and 1% silicon), nickel and calcium oxalate monohydrate. TGA samples between 1-10 mg were monitored for percent weight loss as heated from 25 to 250° C. at 10° C./min in a furnace purged with Helium at ca. 50 mL/min. The percent weight loss to 150° C. is the value reported in Table 16 and the TGA thermogram of detomidine free base in FIG. 18.

Table 16 shows the first “25” peaks
identified in an automatic peak search

| Pos. | Height | FWHM Left | d-spacing | Rel. Int. |
|---|---|---|---|---|
| [°2θ] | [cts] | [°2θ] | [Å] | [%] |
| 9.4369 | 6143.66 | 0.0708 | 9.37201 | 10.89 |
| 10.2822 | 1613.51 | 0.0787 | 8.60336 | 2.86 |
| 13.1267 | 19079.67 | 0.1023 | 6.74475 | 33.81 |
| 16.4182 | 11003.25 | 0.1023 | 5.39925 | 19.50 |
| 16.8720 | 1313.14 | 0.0945 | 5.25502 | 2.33 |
| 18.1807 | 56437.43 | 0.1496 | 4.87959 | 100.00 |
| 18.8002 | 38924.53 | 0.0866 | 4.72018 | 68.97 |
| 18.9037 | 43045.45 | 0.0866 | 4.69456 | 76.27 |
| 20.5659 | 12491.60 | 0.0787 | 4.31875 | 22.13 |
| 21.0058 | 9893.49 | 0.0866 | 4.22929 | 17.53 |
| 21.2170 | 19555.98 | 0.0864 | 4.18419 | 34.65 |
| 21.2634 | 12727.98 | 0.0384 | 4.18555 | 22.55 |
| 23.1638 | 12929.43 | 0.1728 | 3.83676 | 22.91 |
| 24.1591 | 9445.17 | 0.0960 | 3.68089 | 16.74 |
| 24.2191 | 6714.47 | 0.0384 | 3.68103 | 11.90 |
| 24.6326 | 16859.78 | 0.1632 | 3.61120 | 29.87 |
| 25.4240 | 5466.94 | 0.1056 | 3.50055 | 9.69 |
| 25.9866 | 26594.20 | 0.1056 | 3.42603 | 47.12 |
| 26.0536 | 17505.31 | 0.0384 | 3.42587 | 31.02 |
| 26.4132 | 1532.75 | 0.0576 | 3.37165 | 2.72 |
| 28.1107 | 7071.67 | 0.1248 | 3.17180 | 12.53 |
| 28.5554 | 17443.62 | 0.1056 | 3.12340 | 30.91 |
| 29.1749 | 265.43 | 0.1536 | 3.05848 | 0.47 |
| 30.0116 | 2373.54 | 0.0960 | 2.97508 | 4.21 |
| 30.8465 | 598.97 | 0.1152 | 2.89644 | 1.06 |

1. Detomidine or a pharmaceutically acceptable salt thereof, the compound substantially free of iso-detomidine.

2. The detomidine of claim 1, wherein the pharmaceutically acceptable salt of detomidine is detomidine HCl.

3. The detomidine of claim 2, wherein the detomidine HCl is the monohydrate form.

4. The detomidine of claim 2, wherein the detomidine HCl is the anhydrous form.

5. The detomidine any one of claims 1-4, wherein the total amount of impurities is not more than 0.1% area, not more than 0.06% area, or not more than 0.02% area, based on HPLC.

6. The detomidine of any one of claims 1-5, wherein the total amount of impurities is not more than 0.06% area, based on HPLC.

7. The detomidine of any one of claims 1-6, wherein the detomidine or a pharmaceutically acceptable salt thereof is further substantially free of iso-impurity A (((RS)-(3,4-dimethylphenyl)(1H-imidazol-4-yl)methanol)) and/or impurity A ((RS)-(2,3-dimethylphenyl)(1H-imidazol-4-yl)methanol) and/or (2,3-dimethylphenyl)(1H-imidazol-4-yl) methanone.

8. Detomidine or a pharmaceutically acceptable salt thereof, the compound substantially free of iso-detomidine and iso-impurity A (((RS)-(3,4-dimethylphenyl)(1H-imidazol-4-yl)methanol)).

9. Detomidine or a pharmaceutically acceptable salt thereof, the compound substantially free of iso-detomidine and (2,3-dimethylphenyl)(1H-imidazol-4-yl) methanone.

10. Detomidine or a pharmaceutically acceptable salt thereof, the compound substantially free of iso-detomidine, iso-impurity A (((RS)-(3,4-dimethylphenyl)(1H-imidazol-4-yl)methanol)) and (2,3-dimethylphenyl)(1H-imidazol-4-yl) methanone.

11. The detomidine of any one of claims 1-6 and 8-10, wherein the detomidine or a pharmaceutically acceptable salt thereof is further substantially free of impurity A ((RS)-(2,3-dimethylphenyl)(1H-imidazol-4-yl)methanol).

12. The detomidine of any one of claims 1-11, wherein the detomidine or a pharmaceutically acceptable salt thereof is further substantially free of impurity B ((RS)-(1-benzyl-1H-imidazol-5-yl)(2,3-dimethylphenyl)methanol) and/or impurity C (4-[(2,3-dimethylcyclohexyl)methyl]-1H-imidazole).

13. A composition comprising the detomidine of any one of claims 1-12, and a vehicle.

14. A pharmaceutical composition comprising detomidine or a pharmaceutically acceptable salt thereof substantially free of iso-detomidine; and a pharmaceutically acceptable carrier.

15. The pharmaceutical composition of claim 14, wherein the pharmaceutically acceptable salt of detomidine is detomidine HCl.

16. The pharmaceutical composition of claim 15, wherein the detomidine HCl is the monohydrate form.

17. The pharmaceutical composition of claim 15, wherein the detomidine HCl is the anhydrous form.

18. The pharmaceutical composition of any one of claims 14-17, wherein the total amount of impurities is not more than 0.1% area, not more than 0.06% area, or not more than 0.02% area, based on HPLC.

19. The pharmaceutical composition of any one of claims 14-17, wherein the total amount of impurities is not more than 0.06% area, based on HPLC.

20. The pharmaceutical composition of any one of claims 14-19, further substantially free of iso-impurity A (((RS)-(3,4-dimethylphenyl)(1H-imidazol-4-yl)methanol)).

21. The pharmaceutical composition of any one of claims 14-20, further substantially free of (2,3-dimethylphenyl)(1H-imidazol-4-yl) methanone.

22. A pharmaceutical composition comprising detomidine or a pharmaceutically acceptable salt thereof substantially free of iso-detomidine, iso-impurity A (((RS)-(3,4-dimethylphenyl)(1H-imidazol-4-yl)methanol)) and (2,3-dimethylphenyl)(1H-imidazol-4-yl) methanone; and a pharmaceutically acceptable carrier.

23. A pharmaceutical composition comprising detomidine or a pharmaceutically acceptable salt thereof substantially free of iso-detomidine and iso-impurity A (((RS)-(3,4-dimethylphenyl)(1H-imidazol-4-yl)methanol)); and a pharmaceutically acceptable carrier.

24. A pharmaceutical composition comprising detomidine or a pharmaceutically acceptable salt thereof substantially free of iso-detomidine and (2,3-dimethylphenyl)(1H-imidazol-4-yl) methanone; and a pharmaceutically acceptable carrier.

25. The pharmaceutical composition of any one of claims 13-24, further substantially free of impurity A ((RS)-(2,3-dimethylphenyl)(1H-imidazol-4-yl)methanol).

26. The pharmaceutical composition of any one of claims 13-25, further substantially free of impurity B ((RS)-(1-benzyl-1H-imidazol-5-yl)(2,3-dimethylphenyl)methanol) and/or impurity C (4-[(2,3-dimethylcyclohexyl)methyl]-1H-imidazole).

27. A process for the preparation of a monohydrate form of detomidine HCl substantially free of iso-detomidine comprising
a. crystallizing a monohydrate form of detomidine HCl from an aqueous solution of an anhydrous form of detomidine HCl which comprises iso-detomidine; wherein the crystallization takes place by cooling the aqueous solution to a temperature of from about 0° C. to 31° C., optionally in the presence of solid detomidine base or detomidine HCl, until a slurry is formed, and
b. collecting the monohydrate form of detomidine HCl substantially free of iso-detomidine.

28. A process for the preparation of a monohydrate form of detomidine HCl substantially free of iso-detomidine comprising
a. treating an aqueous solution of an anhydrous form of detomidine HCl which comprises iso-detomidine with active carbon;
b. converting the detomidine HCl to detomidine base to form an aqueous solution of detomidine base;
c. crystallizing the monohydrate form of detomidine HCl from the aqueous solution of detomidine base by adding a sufficient amount of HCl, wherein the crystallization takes place by cooling the aqueous solution to between about 3° C. and 37° C., optionally in the presence of solid detomidine HCl, until a slurry is formed, and
d. collecting the monohydrate form of detomidine HCl substantially free of iso-detomidine.

29. The process of claim 27 or 28, wherein the HCl:detomidine base ratio (mole:mole) is about 1 to about 1.5.

30. The process of claim 29, wherein the HCl:detomidine base ratio (mole:mole) is about 1.5.

31. The process of any one of claims 27-30, wherein the water:detomidine (V/wt) HCl ratio is about 2-3.

32. The process of any one of claims 27-31, wherein the crystallization of the monohydrate form of detomidine HCl from an aqueous solution occurs at a dissolution temperature of between about 35° C.-50° C.

33. The process of any one of claims 27-32, wherein the crystallization initiation temperature is about 30° C.-45° C.

34. The process of any one of claims 27-33, wherein the monohydrate form of detomidine HCl has a mean crystal size of 0.3 to 0.7 mm.

35. The process of claim 34, wherein the shape of the crystals of the monohydrate form of detomidine HCl is rod like and/or prism like.

36. The process of any of claims 27-35 wherein the monohydrate form of detomidine HCl has a water content of about 7.5% as determined by Karl Fisher analysis.

37. The monohydrate form of detomidine HCl obtained by a process of any one of claims 24-33.

38. A composition comprising the monohydrate form of detomidine HCl obtained by a process of any one of claims 27-36; and a vehicle.

39. A pharmaceutical composition comprising the monohydrate form of detomidine HCl obtained by a process of any one of claims 27-36; and a pharmaceutically acceptable carrier

40. A method of treating a human subject in need thereof, comprising administering to the human subject the pharmaceutical composition of any one of claims 14-26 and 39.

41. The method of claim 40, wherein the human subject is in need of an analgesic.

42. A process for validating a batch of a monohydrate form of detomidine HCl drug substance, comprising
a. determining the amount of iso-detomidine in a sample of the batch, and
b. validating the batch for distribution only if the sample of the batch contains not more than 0.01% area of of iso-detomidine and/or iso-impurity A relative to detomidine.

43. The detomidine of claim 1, which is detomidine free base.

44. The detomidine free base of claim 43, having 2 theta values comprising at least one of the values as shown in Table 16, herein.
